(19) United States
(12) Patent Application Publication
(10) Pub. No.: US 2011/0034916 A1
(43) Pub. Date: Feb. 10, 2011

(54) NANOPARTICLE-MEDIATED MICROWAVE TREATMENT METHODS

(76) Inventors: Alexis Te, Manhasset, NY (US); Carl Batt, Groton, NY (US); Diego Rey, Palo Alto, CA (US)

Correspondence Address: MARJAMA MULDOON BLASIAK & SULLIVAN LLP, 250 SOUTH CLINTON STREET, SUITE 300, SYRACUSE, NY, 13202, US

(21) Appl. No.: 12/936,647

(22) PCT Filed: Apr. 6, 2009
(86) PCT No.: PCT/US2009/039652

§ 371 Date: Oct. 6, 2010

Related U.S. Application Data
(60) Provisional application No. 61/043,472, filed on Apr. 9, 2008.

(51) Int. Cl. A61B 18/18; A61K 9/14; A61P 35/00; B82Y 5/00

(52) U.S. Cl. 606/33; 424/490; 977/773; 977/915

(57) ABSTRACT

A method is provided for using magnetic nanoparticles to enhance microwave therapies for treating cells and tissues. The nanoparticles are designed to transduce microwave radiation into heat and furthermore, the nanoparticles may include specific tissue targeting and other functionality for enhancing in situ effects. In one embodiment, nanoparticles are introduced into a tissue system and a microwave field is applied. The nanoparticles react to the microwave energy by releasing heat thus heating the tissue and inducing hyperthermia (below 50° C.) or thermotherapy (above 50° C.). The nanoparticles can be designed for optimal heat production response at specific microwave frequencies and/or ranges of microwave frequencies where these frequencies may span the entire microwave spectrum, namely 300 MHz (310⁸ Hz) to 300 GHz (310¹¹ Hz).

### CROSS-REFERENCE TO RELATED APPLICATIONS

This application claims priority to and the benefit of co-pending U.S. provisional patent application Ser. No. 61/043,472, entitled “Nanoparticle-mediated microwave thermotherapy and tissue treatment methods based thereon,” filed 9 Apr. 2008, which is incorporated herein by reference in its entirety.

### 1. TECHNICAL FIELD

The present invention relates to magnetic nanoparticles and nanoparticle-mediated microwave treatment methods. The invention also relates to methods for treatment of tumors and cancers using nanoparticle-mediated microwave thermotherapy. The invention further relates to systems for administering nanoparticle-mediated microwave treatment.

### 2. BACKGROUND OF THE INVENTION

### 2.1 Benign Prostate Hyperplasia (BPH)

A healthy human male prostate slightly larger than a walnut and commonly increases in size due to aging. More than half of the men in the United States between the ages of 60 and 70 and as many as 90 percent between the ages of 70 and 90 have symptoms of Benign Prostate Hyperplasia (BPH), also known as or benign prostatic hypertrophy or enlarged prostate (Verhamme K M, D. J., Bleumink G S. Incidence and prevalence of lower urinary tract symptoms suggestive of benign prostatic hyperplasia in primary care—the Triumph project. European Urology, 2002. 42(4): p. 323-328). In BPH, the enlarged prostate presses against the urethra and bladder leading to symptoms of urinary hesitancy, frequent urination, increased risk of urinary tract infections and urinary retention (Verhamme K M, D. J., Bleumink G S, Incidence and prevalence of lower urinary tract symptoms suggestive of benign prostatic hyperplasia in primary care—the Triumph project. European Urology, 2002. 42(4): p. 323-328).

The cause of BPH is not known although it is thought that it may be due to hormone activity; the prostate first grows naturally through a period of 12 months during puberty and this is probably related to levels of the sex hormone, testosterone (Verhamme K M, D. J., Bleumink G S. Incidence and prevalence of lower urinary tract symptoms suggestive of benign prostatic hyperplasia in primary care—the Triumph project. European Urology, 2002. 42(4): p. 323-328),

The anatomy and functions of the prostate are well known in the art. Exiting the bladder is the urethra, which is connected to the bladder by the bladder neck, also known as the internal sphincter that is composed of thickened muscle fibers that tighten to hold urine. Immediately following the bladder neck, adjacent to the rectum and surrounding the urethra is the prostate. The prostate is a gland that functions to secrete and store a clear, slightly basic fluid that makes up 10-30% of the seminal fluid. The prostate also contains smooth muscle tissue that it uses to help expel semen into the urethra during ejaculation. The rest of the seminal fluid is produced by the seminal vesicles, which are a pair of glands on the posterior surface of the bladder that secrete proteins, enzymes, fructose, and fatty-acid derivatives necessary for sperm. Sperm are also emptied into the urethras near the prostate gland and through the two vas deferens that originate from the testes. Below the prostate is the external sphincter that also serves to retain urine. The urethra then continues along the penis through which semen and urine exits the body.

### 2.2. Prostate Cancer

Prostate cancer is the most commonly diagnosed cancer in men and the second leading cause of cancer deaths in men after lung cancer. It is estimated to be found in as many as half of all men over the age of 70 and in almost all men over the age of ninety. Since the discovery of the blood test for Prostate Specific Antigen (PSA) in the 1980's, prostate cancer can now be detected at a much earlier stage.

In 1999, there were over 250,000 new cases of prostate cancer with 45,000 deaths. The average age of diagnosis is 72 years and 95% of cases are diagnosed between the ages of 45-89. The incidence of prostate cancer varies among different ethnicities. The incidence is highest in African Americans and lowest in Asian Americans. Mortality from prostate cancer has slowly risen over the last 10 years which is likely attributable to the fact that the American population is aging and experiencing less cardiovascular mortality.

There are several staging systems to categorize the levels of prostate cancer. The most widely accepted system is the TNM classification. Stage I—(T1)—Tumor remains confined to the prostate and is too small to be detected on DRE. This is an incidentally found cancer either by an elevated PSA or found after a transurethral resection of the prostate. Stage II—(T2)—Tumor is still confined to the prostate, but is now large enough to be felt on DRE. Stage III—(T3)—The prostate cancer has spread through the prostatic capsule and may involve locally surrounding tissues such as the seminal vesicles. Stage IV—(T4)—Metastatic prostate cancer in which the cancer involves lymph nodes or bony sites or other organs such as the liver or lungs.

Like BPH, prostate cancer is a tumor of the prostate gland except that it is malignant and can lead to metastatic disease and death. Current curative therapy is dependent on stage at time of treatment, and is best attained at time when disease is localized or Stage I and II. Curative therapy aimed to either remove or destroy malignant prostate tissue. Surgical therapy or radical prostatectomy removes the entire prostate, and carries well know surgical risks such as bleeding, pulmonary embolus, incontinence and impotence. Minimally invasive therapies include radiation therapy, cryotherapy and high intensity focused ultrasound. All aim to cure cancer by destroying tumor tissue within the prostate gland. All these therapies have risk of: incontinence, impotence, stricture formation, and fistulas. Radiation therapy carries a risk of secondary cancers such as bladder tumors. While, these therapies are minimally invasive, none are currently considered outpatient or “office” procedures.

### 2.3 Transurethral Microwave Thermotherapy (TUMT)

Transurethral microwave thermotherapy (TUMT) is to common treatment for BPH symptoms that consists of a catheter-based system containing a microwave antenna used to deliver microwave radiation from the urethra and into the prostate tissue. The device delivers microwave radiation to the prostate to achieve intraprostatic temperatures sufficient for causing tissue necrosis and for the purpose of dilating the prostatic urethra.

Modern TUMT devices are designed on the premise that high intraprostatic temperatures are needed for optimal treatment. In addition, specific targeting, of obstructive intraprostatic tissue is critical, so as not to damage non-target areas such as the rectum, the urinary sphincters, and the penis. Nonspecific heating could lead to serious complications within the patient and could also limit the effectiveness of TUMT devices due to programmed safety mechanisms that cause the device to shut down (Larson T R, B. M., Tri J L, Whitlock S V, Contrasting heating patterns and efficiency of the Prostratron and Targis microwave antennae for thermal treatment of benign prostatic hyperplasia. Urology 1998. 51(6): p. 908-915).

All TUMT devices use a catheter-based system that contains a microwave antenna used to deliver microwave radiation from the urethra and into the prostate tissue. The catheter is maintained in proper position by the use of a balloon that is inflated in the bladder. The device often employs a cooling system in which water flows through the inside at the catheter in order to protect the urethra. This cooling system along with the design of the antenna, the power level, and duration of treatment allows for generating intraprostatic temperatures sufficient for tissue necrosis and also targets a specified heating geometry. The Boston Scientific Prolieve TUMT device (REF), for example, employs a balloon along the length of the microwave antenna that serves to help dilate the prostatic urethra.

Possible side effects as well as the efficacy of TUMT systems are determined by the capability of the device to effectively target sufficient heating within the prostate. TUMT devices are equipped with temperature probes that monitor areas such as the rectum, penis, and urethra and have safety mechanisms that shutdown the microwave radiation if preset temperature limits for these areas are exceeded. The most vulnerable areas that can be affected due to stray heating are the external sphincter, the bladder neck, the penis, and the rectum. Damage to the urinary sphincters can lead to urinary incontinence, damage to the penis can lead to loss of erectile function, and damage to the rectum and specifically the anus can lead to fecal incontinence.

### 2.4 Use of Nanoparticles in Microwave Applications

Gold nanoparticles have been used to remotely heat and dissolve proteins in vitro (Neus G. Bastus, M. J. K., Roger Amigo, Dolors Grillo-Bosch, Eyleen Araya, Antonio Turiel, Amilcar Labarta, Ernest Giralt, Victor F. Fumes, Gold nanoparticles for selective and remote heating of β-amyloid protein aggregates. Materials Science and Engineering C, 2007. 27: p. 1236-1240). Protein solutions with and without gold nanoparticles were subjected to microwave irradiation. The study showed that the particles produced heat via microwave irradiation without heating the aqueous solution itself. The Neus et al. study suggested that their method could be extended to a number of systems in vitro where it may be desirable to remove proteins and other aggregates involved in different pathologies.

Limited uses for magnetic nanoparticles and microwave as a therapy have also been disclosed in U.S. Pat. Nos. 6,165,440, 6,955,639 B2 and 7,074,175 B2. U.S. Pat. No. 6,955,639 B2 mentions the use of this technique only on ex-vivo tissue samples, since the conditions and methods disclosed produced too much heating of non-target tissue tear use in vivo.

Outside of clinical in vivo applications, microwaves have been used to induce heating from nanoparticles, although these applications studied this effect in heating of solutions for industrial applications. Nanoparticles such as carbon nanotubes, carbon black, carbon nanotube-iron particle complexes have been used (A. Wadhawan, D. G., J. M. Perez, Nanoparticle-assisted microwave absorption by single-wall carbon nanotubes. Applied Physics Letters, 2003. 83(13): p. 2683-2685) and magnetic nanoparticles (Arnold Holzwarth, J. L., T. Alan Hatton, Paul E. Laibinis, Enhanced Microwave Heating of Nonpolar Solvents by Dispersed Magnetic Nanoparticles. Industrial & Engineering Chemistry Research, 1998. 37: p. 2701-2706).

Nanoparticle-mediated hyperthermia and thermotherapies have also been studied and have resulted in clinical trials and commercial applications. None, however, has employed microwave radiation in vivo in these processes. Carbon nanotubes, gold and gold containing nanoparticles and magnetic nanoparticles have been previously employed in clinically motivated heating applications (Nadine Wong Shi Kam, M. O. C., Jeffrey A. Wisdom, Hongjie Dai, Carbon nanotubes as multifunctional biological transporters and near-infrared agents for selective cancer cell destruction. PNAS, 2005 102 (33): p. 11600-11605; Eijiro Miyako, H. N., Ken Hirano, Yoji Makita, Ken-ichi Nakayama, Takahiro Hirotsu, Near-infrared laser-triggered carbon nanohorns for selective elimination of microbes. Nanotechnology 2007. 18: p. 475103-475110; Xiaohua Huang, I. H. E.-S., Wei Qian, Mostafa A. El-Sayed, Cancer Cell Imaging and Photothermal Therapy in the Near-Infrared Region by Using Gold Nanorods. JACS, 2006. 128: p. 2115-2120; Akira Ito, Kazuyoshi Kondo; Masashige Shinkai, Hiroyuki Honda, Kazuhiko Matsumoto, Yoshiaki Saida, Takeshi Kobayashi, tumor regression by combined immunotherapy and hyperthermia using magnetic nanoparticles in an experimental subcutaneous murine melanoma. Cancer Science, 2003. 94(3): p. 308-313).

Gold nanoshells have been used to mediate near-infrared thermal therapy of tumors under magnetic resonance guidance (L. R. Hirsch, R. J. S., J. A. Bankson, S. R. Sershen, B. Rivera, R. E. Price, J. D. Hazle, N. J. Halas, J. L. West, Nanoshell-mediated near-infrared thermal therapy of tumors under magnetic resonance guidance. PNAS 2003. 100 (23): p. 13549-13554). This technology, however, is specific to laser irradiation in the near-infrared. Near-infrared is a spectrum, of light in which tissue is fairly transparent (i.e., does not heat). Limitations of this method include the instrumentation that is required to deliver laser light for treatment and the penetration depth of the light into the targeted tissue.

Magnetic nanoparticles are an extensively studied nanomaterial in in vivo heating applications and have gone through significant clinical trials for the treatment of prostate cancer (Manfred Johannsen. U. G., Burghard Thiesen, Kasra Taymoorian, Chie Hee Cho, Norbert Waldofner, Regina Scholz, Andreas Jordan, Stefan A. Loening, Peter Wust, Thermotherapy of Prostate Cancer Using Magnetic Nanoparticles: Feasibility, imaging, and Three-Dimensional Temperature Distribution, Eeuropean Urology, 2007. 52: p. 1653-1662), MagForce Nanotechnologies AG of Berlin, Germany is commercializing these materials and treatment systems for hyperthermia and thermotherapy treatments of cancer www.magforce.de)

Dann (U.S. Pat. No. 6,148,236 entitled Cancer Therapy Treatment System, issued Nov. 14, 2000) describes the use of an “energy-emitting element” that comprises “a seed that elevates in temperature in the presence of a magnetic field.” These seeds are inserted in a cartridge, which is implanted in target tissue. Seeds can be composed of a radioactive material or a non-radioactive material that elevates in temperature when placed in a magnetic field (Col. 3, lines 11-18).

Ivkov et al. (US 2008/0213382 A1 entitled Thermotherapy Susceptors and Methods of Using Same, published Sep. 4, 2008) describes the use of nanoparticles to enhance thermotherapy with the main focus of kilo-hertz frequency rage alternating magnetic fields (kHz AMF). Ivkov et al. also discloses the use of microwave radiation as a possible alternative to kHz AMP, however, the design and characteristics of nanoparticles for use with microwaves is not disclosed.

None of the currently known methods that use magnetic nanoparticles for in vivo heating applications, however, use microwaves for in vivo heating. Magnetic nanoparticle applications have employed kilohertz-range alternating electromagnetic fields to cause the particles to emit heat from hysteresis losses. All other nanoparticle heating applications have employed near infrared laser radiation for exciting the particles to emit heat.

The studies discussed above disclose the use nanoparticles for enhanced hyperthermia and thermotherapies with alternating magnetic fields in the kilo-hertz frequency range. These previous studies did not investigate, however, whether enhanced heating from nanoparticles can be achieved in vivo, i.e., whether microwave irradiation produces more heating in tissue targeted with nanoparticles than in tissue alone. They also did not investigate whether the heating differential achieved by microwaves is sufficient for therapeutic applications while maintaining a safe temperature in non-target tissue.

There is therefore, a need in the art for methods for focusing microwave thermotherapy within controlled areas inside organs and tissues, and in particular, within the prostate. There is also a need in the art for methods for focusing microwave thermotherapy with control down to the cellular level. There is further a need in the art for methods for using magnetic nanoparticle applications that employ focused microwaves for exciting the particles to emit heat. Such methods of focused microwave thermotherapy could be used in the treatment of BPH, prostate cancer and other types of cancer. Citation or identification of any reference in Section 2, or in any other section of this application, shall not be considered an admission that such reference is available as prior art to the present invention.

### 3. SUMMARY OF THE INVENTION

A method is provided thr using nanoparticles to enhance microwave therapies (e.g., thermotherapy) for treating cells and tissues in vivo. The method can employ lower-than-normal microwave powers, thereby minimizing risks for side-effects while still allowing for the localized and accurate delivery of effective thermal doses to targeted tissue.

The nanoparticles are designed to transduce microwave radiation into heat. The microwave-active nanoparticles can be designed for optimal heat production response at specific microwave frequencies and/or ranges of microwave frequencies where these frequencies may span the entire microwave spectrum, namely 300 MHz (3×108 Hz) to 300 GHz (3×1011 Hz).

The nanoparticles can include specific tissue targeting and other functionality for enhancing in situ effects. The nanoparticles can be linked to chemical and/or biochemical moieties which hind specifically to the target tissue.

In one embodiment, nanoparticles are introduced into a tissue (or organ) system and a microwave field is applied. The nanoparticles can be introduced or administered intravenously, intra-arterially, intracavitary, intraspinal, lymphatic, or locally such as direct injection or physical placement via percutaneous, via natural orifice pathways (oral, anal, topical, etc.) to achieve specific loading in and around the target tissue.

The nanoparticles react to the microwave energy by releasing heat thus heating the tissue and inducing hyperthermia (below 50° C.) or thermotherapy (above 50° C.).

In one aspect, a method for treating a cell or tissue of interest in a subject in need thereof is provided. The methods can comprise the steps of:

introducing microwave-active nanoparticles into the cell or tissue; and

applying a microwave, field,

wherein:

the microwave-active nanoparticles react to microwave energy of the microwave field, by releasing heat, and

the tissue is heated, thereby inducing hyperthermia or thermotherapy in the tissue.

In one embodiment, the subject is an animal. In another embodiment, the animal is a human.

In another embodiment, the cell or tissue is selected from the group consisting of prostate tissue, tumor tissue (e.g., benign or cancerous), solid cancer tissue, non-solid cancer tissue, leukemic cells, bone marrow cancer cells, lymphogenic cancer tissue, bladder tissue, uterine tissue, and uterine fibroid tissue.

In another embodiment, the step of applying the microwave field is selected from the group consisting of applying transurethrally, applying transrectally, applying transcutaneously, and applying directly via surgery (e.g., open surgery or other suitable surgical procedure known in the art).

In another embodiment, the nanoparticle is designed or tuned to interact with Microwaves such that the nanoparticle is more loss y in the presence of microwaves than the cells or tissue of interest are. In another embodiment, the nanoparticle is functionalized with to functional coating.

In another embodiment, the functional coating is a biocompatibility coating, an inorganic coating, or a hydrophilic coating.

In another embodiment, the functional coating can comprise a targeting wherein the targeting ligand targets the cell or tissue of interest.

In another embodiment, the functional coating can comprise a material that promotes nanoparticle aggregation within the cell or tissue of interest.

In another embodiment, the nanoparticles have diameters of 1-500 nm.

In another aspect, a method for treating cancerous tissue in a subject in need thereof is provided. The method can comprise the steps of:

introducing microwave-active nanoparticles into the cancerous tissue; and

applying a microwave field,

wherein:

the microwave-active nanoparticles react to microwave energy of the microwave field by releasing heat, and

the cancerous tissue is heated, thereby inducing hyperthermia in the cancerous tissue.

In one embodiment, the subject is an animal. In another embodiment, the animal is a human.

In another embodiment, the cell or tissue is selected from the group consisting of prostate tissue, tumor tissue (e.g., benign or cancerous), solid cancer tissue, non-solid cancer tissue, leukemic cells, bone marrow cancer cells, lymphogenic cancer tissue, bladder tissue, uterine tissue, uterine fibroid tissue.

In another embodiment, the step of applying the microwave field is selected from the group consisting of applying transurethrally, applying transrectally, applying transcutaneously, and applying directly via surgery (e.g., open surgery or other suitable surgical procedure known in the art.

In another embodiment, the nanoparticle is designed or tuned to interact with microwaves such that the nanoparticle is more loss in the presence of microwaves than the cells or tissue of interest are. In another embodiment, the nanoparticle is functionalized with a functional coating,

In another embodiment the functional coating is a biocompatibility coating, an inorganic coating, or a hydrophilic coating.

In another embodiment, the functional coating can comprise a targeting ligand, wherein the targeting ligand targets the cell or tissue of interest.

In another embodiment, the functional coating can comprise a material that promotes nanoparticle aggregation within the cell or tissue of interest.

In another embodiment, the nanoparticles have diameters of 1-500 nm.

In another aspect, a method for aggregating nanoparticles in a cell or tissue of interest in a subject in need thereof is provided. The method can comprise the step of introducing nanoparticles into the cell or tissue, wherein the nanoparticles are functionalized with a functional coating.

In another embodiment, the method can further comprise the step of applying a source of radiating energy.

In another embodiment, the functional coating is a biocompatibility coating, an inorganic coating, or a hydrophilic coating.

In another embodiment, the functional coating comprises a targeting ligand, wherein the targeting ligand targets the cell or tissue of interest.

In another embodiment, the functional coating can comprise a material that promotes nanoparticle aggregation within the cell or tissue of interest.

In another embodiment, the nanoparticles have diameters of 1-500 nm.

In another embodiment, the nanoparticle is designed or tuned to interact with energy from a radiating energy source.

In another embodiment, the nanoparticle is tuned to interact with microwaves such that the nanoparticle is more lossy in the presence of microwaves than the cells or tissue of interest are.

In yet another aspect, a nanoparticle for treating a cell or tissue of interest is provided. In one embodiment, the nanoparticle is designed to be tuned to interact with microwaves such that the nanoparticle is more lossy in the presence of microwaves than the cells or tissue of interest are, and is functionalized with a functional coating.

In another embodiment, the functional coating is a biocompatibility coating, an inorganic coating, or a hydrophilic coating.

In another embodiment, the functional coating can comprise a targeting ligand, wherein the targeting ligand targets the cell or tissue of interest.

In another embodiment, the functional coating can comprise a material that promotes nanoparticle aggregation within the cell or tissue of interest.

In another embodiment, the nanoparticle has a diameter of 1-500 nm.

In yet another aspect, a system is provided for controlling effects of a field of microwave radiation in a cell or tissue of interest in a subject in need thereof. The system can comprise:

a source of microwave radiation;

an electronic system for monitoring, of the microwave radiation;

a system for delivery of the microwave radiation to the cell or tissue;

microwave-active nanoparticles that absorb the microwave radiation:

an injection or administration system for administration of the nanoparticles;

wherein:

the microwave-active nanoparticles react to microwave energy of the field of microwave radiation by releasing heat, and

the cell or tissue is heated, thereby inducing hyperthermia or thermotherapy in the cell or tissue,

and whereby the effects of the field of microwave radiation are controlled.

In one embodiment, the effects are controlled by using the methods of the invention to alter hydration of a biological target. In another embodiment, the microwave field of radiation is altered by modifying antenna design. In another embodiment, the effects are controlled by using direct cooling or application of pressure to biological target, e.g., in designs for transurethral, transrectal and other natural orifice route of entry as well as transcutaneous, and other routes of open surgical access.

The methods of the invention are advantageous in that they can easily integrate established, advanced, clinically approved and routine treatment methods. Furthermore, an already well-established infrastructure for administering microwave radiation exists. The enhanced microwave therapy methods (e.g., thermotherapy) of the invention can also be adapted to ablate unwanted tissues or cells ex vivo.

### 4. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated or enlarged to facilitate an understanding of the invention.

FIGS. 1A-C. Oleic acid capped Fe₃O₄ particles: A) TEM micrograph. B) Size distribution from light scattering; data. C) Magnetic characteristics from hysteresis data. See Section 6 for details.

FIGS. 2A-B. Illustration of nanoparticle functionalization. A) Oleic acid-capped nanoparticles, then phospholipid coating, and protein conjugation to the phospholipid coat. B) zoomed in view of illustration of functionalization.

FIGS. 3A-B, Phospholipid capped Fe₃O₄ particles: A) TEM micrograph. B) Size distribution from light scattering data. See Section 6 for details.

FIG. 4. Phantom experimental setup. See Section 6 for details.

FIGS. 5A-B. A33 targeting, single chain variable, fragment antibody conjugation to phospholipid coated nanoparticles. A) Dot blot demonstrating the presence of antibody on the nanoparticles, B) cell culture experiments targeting A33 antigen expressing SW1222 colon cancer cells (top) and not targeting A33 antigen non-expressing HT29 cells (bottom). See Section 6 for details.

FIG. 6. Ex vivo experiments of nanoparticle enhanced TUMT in a bull prostate. See Section 6 for details.

FIGS. 7A-D. In vivo experiments of nanoparticle enhanced TUMT in canine prostate. For graphical data: solid square: temperature probe 1, empty square: temperature probe 2, solid circle: temperature probe 3, empty circle: temperature probe 4, solid upwards pointing triangle: TUMT coolant water, empty upwards pointing triangle: MDS, solid downward pointing triangle: rectal temperature probe, dashed line: TUMT microwave power. A) Illustration of nanoparticle injection and temperature probe positioning. B) Experiment on dog 1. C) experiment on dog 2, D) experiment on dog 3. See Section 6 for details.

### 5. DETAILED DESCRIPTION OF THE INVENTION

A method for treatment of tissue in vivo using nanoparticles to mediate and enhance microwave therapies is provided. The method is based on the discovery by the inventors that microwave-active nanoparticles can be used for focusing microwave thermotherapy within controlled areas inside organs and tissues, e.g., the prostate, and with control down to the cellular level. Such microwave treatments using nanoparticles generate more heating of target tissue than does microwave treatment of the target tissue without the nanoparticles. An advantage of using particles of this size is that heat transfer is rapid to surrounding tissues and temperature gradients are reduced. The method also can employ lower-than-normal microwave powers, thereby minimizing risks for side-effects while still allowing for the localized and accurate delivery of effective thermal doses to targeted tissue.

Nanoparticles used in the methods of the invention are designed to transduce microwave radiation into heat. In one embodiment, nanoparticles are used that include specific tissue targeting and other functionality for enhancing in situ effects.

In one embodiment, nanoparticles are introduced into a tissue system and a microwave field is applied. The nanoparticles react to the microwave energy by releasing heat thus heating the tissue and inducing hyperthermia (below 50° C.) or thermotherapy (above 50° C.). The microwave-active nanoparticles can be designed for optimal heat production response at specific microwave frequencies sand or ranges of microwave frequencies where these frequencies may span the entire microwave spectrum, namely 300 MHz (3×108 Hz) to 300 GHz (3-1011 Hz),

For clarity of disclosure, and not by way of limitation, the detailed description of the invention is divided into the subsections set forth below.

### 5.1. Nanoparticles for Use in Microwave Thermotherapy

A nanoparticle for treating a cell or tissue of interest is provided. In one embodiment, the nanoparticle is designed or tuned to interact with microwaves such that the nanoparticle is more lossy in the presence of microwaves than the cells or tissue of interest are. The nanoparticle is designed (tuned) to react to a specific microwave frequency. Thus, in the presence of cells or tissue being heated at a specific power (e.g., low power) at a specific microwave frequency that generates a low level of heat in the cell/tissue environment, the nanoparticle will react to the microwave field to become hotter (release more heat) than the surrounding cells or tissue. Because not all tissues will react in same manner (become heated) to the same microwave frequency, different tissues will need to be exposed to different microwave frequencies to attain a desired microwave effect. Specific nanoparticles can be tuned to these microwave frequency to provide the needed temperature profile.

The nanoparticle can be functionalized with a functional coating. The functional coating can be, for example, a biocompatibility coating, an inorganic coating, or a hydrophilic coating. The functional coating can comprise a targeting ligand, wherein the targeting ligand targets the cell or tissue of interest. In other embodiments, the functional coating can comprise a material that promotes nanoparticle aggregation within the cell or tissue of interest. Examples of such materials are known in the art (L. Josephson, et al., Angew. Chem. Int. Ed. 2001, 40, No. 17; Y. Jun, et al., J. Am. Chem. Soc. 2005, 127, 5732-5733; J. M. Perez, et al., Nature Biotech 2002, 20, 816-820; A. Tsourkas, et al., Angew. Chem. Int. Ed. 2004, 43, 2395-2399).

One advantage of using nanoparticles is that heat transfer is rapid to the surrounding medium and reduces large temperature gradients when particles are homogeneously distributed. The ‘thermal bystander effect’, a hyperthermia-induced deep infiltration of nanoparticles in tissue, facilitates even distribution of locally injected nanoparticles (Jordan A, W. P., Scholz R, Effects of magnetic fluid hyperthermia (MFH) on C3H mammary carcinoma in vivo. International Journal of Hyperthermia. 1997. 13: p. 587-605). Another advantage is that nanoparticles can be implemented into existing clinical infrastructure. A further advantage is that functionalized particles can be designed such that they are taken up selectively into specific cells thus allowing cell specific and intracellular treatment giving cellular-level control of the therapy.

Lower than normal microwave powers can be used, thereby minimizing the risk for side-effects while still allowing for the localized and accurate delivery of effective thermal doses to targeted tissue thus overcoming drawbacks and limitations of microwave therapies.

Metal nanoparticles useful in absorbing electromagnetic radiation are known in the art. Any nanomaterial or nanoparticle that responds to microwaves by emitting heat can be employed in the methods of the invention. This includes, but is not limited to, carbon nanotubes, metal nanoparticles, and magnetic nanoparticles. In a preferred embodiment, magnetic nanoparticles are used, since they frequently have greater microwave absorption characteristics than metals and polar liquids. A nanotube can have a diameter of about 1 to about 10 nm and a length of about 100 to about several thousand nm. A nanoparticle can have a diameter from about 0.1 nm to about 1000 nm. In a specific embodiment, the nanoparticle has a diameter of 1-500 nm.

For example, U.S. Pat. No. 6,955,639 (entitled Methods of enhancing radiation effects with metal nanoparticles. Hainfeld et al., Oct. 18, 2005) discloses metal nanoparticles 0.5 to 400 nm in diameter and methods for using them to enhance the dose and effectiveness of x-rays in ablating a target tissue such as tumor.

Characteristics of a magnetic nanoparticle, such as its material composition, size, and shape, can affect its heating properties and its sequestration by various issue types. Many of these characteristics can be designed, using art-known methods, to tailor the heating properties for a particular set of conditions found within a tissue type. For example, principles for designing magnetic particles tailored for specific heating properties and tissue types are disclosed in U.S. Pat. No. 7,074,175 (entitled “Thermotherapy via targeted delivery of nanoscale magnetic particles,” Handy et al., Jul. 11, 2006) at, inter alia, cols. 10-16.

As particles, magnetic materials exhibit heating from microwave absorption due to the effect of ferromagnetic resonance (Griffiths, Anomalous High-Frequency Resistance of Ferromagnetic Metals. Nature, 1946. 158(4019): p. 670-671; C. Surig, K. A. H., Interaction effects in particulate recording media studied by ferromagnetic resonance. Journal of Applied Physics, 1996. 80(6): p. 3427-3429). The magnetic dipoles within the magnetic nanoparticles can be excited by microwave irradiation to process and the coupling between the magnetic dipoles and the microwave field transforms the radiation energy into heat. The energy conversion is at its maximum when the applied microwave frequency is at the resonant frequency of the particle. The resonant frequency depends on the magnetic properties of the particle material as well as on the size and shape of the particle. This dependence provides a way of modulating the microwave absorption by the particles (Arnold Holzwarth, J. L., T. Alan Hatton, Paul E. Laibinis, Enhanced Microwave Heating of Nonpolar Solvents by Dispersed Magnetic Nanoparticles. Industrial & Engineering Chemistry Research, 1998. 37: p. 2701-2706).

A particle with higher saturation magnetization as governed by the particle's material bulk properties absorbs greater amounts microwave energy (Arnold Holzwarth, J. L., T. Alan Hatton, Paul E. Laibinis, Enhanced Microwave. Heating of Nonpolar Solvents by Dispersed Magnetic Nanoparticles. Industrial & Engineering Chemistry Research, 1998. 37: p. 2701-2706). Microwave energy absorption also increases when a particles resonant frequency is tuned to the applied microwave frequency and this resonant frequency decreases with decreasing particle size (Griffiths, J. H. E., Anomalous High-Frequency Resistance of Ferromagnetic Metals. Nature, 1946, 158(4019); p. 670-671; (Surig, K. A. H., Interaction effects in particulate recording media studied by ferromagnetic resonance. Journal of Applied Physics, 1096. 80(6): p. 3427-3429).

### 5.2 Nanoparticle Synthesis

Synthesis of monometallic magnetic nanoparticles such as cobalt, iron, nickel, and the oxides of these metals are known in the art. Metal alloys with additional heterometals have been synthesized. The heterometals provide greater control on the magnetic properties of these materials (Park, H. Y. J; Seo, S; Kim, K; Yoo, K. H., 2008, Multifunctional Nanoparticles for Photothermally Controlled Drug Delivery and Magnetic Resonance Imaging Enhancement, Small 4(2); 192-196; Digital Object identifier (DOI):10.1002/smll.200700807). Using these methods known in the art, rational syntheses can be easily carried out by the ordinarily skilled practitioner to build a library of nanomaterials that can strongly absorb microwave radiation at specific frequencies.

In a specific embodiment, nanoparticles used in nanoparticle-mediated treatment methods such as microwave thermotherapy can be metal-doped magnetism-engineered iron oxide (MEIO) nanoparticles of spinel MFe₂O₄ where M is +2 cation of Mn, Fe, Co or Ni (Lee, J. H., Y M; Jun, J W; Jang, J T; Cheon, J, Artificially engineered magnetic nanoparticles for ultra-sensitive molecular imaging; Nature medicine. Nature Medicine, 2007. 13(1): p. 95-99). Using the methods described, in Lee et al., magnetism-engineered iron oxide (MEIO) nanoparticles can be synthesized that possess exceptionally high and tunable nanomagnetism. The artificial synthetic protocol of Lee et al., performed under high temperature in an organic medium, can be used to obtain high-quality n an particles in which size, uniformity, single crystallinity, stoichiometry and high magnetism are controlled and enhanced. Lee et al. describes methods by which a series of spinel nanoparticles that possess a variety of metallic dopants with distinct magnetic spin magnitudes can be characterized and assessed.

Particles can be synthesized following other published protocols known in the art. For example, 4-nm Fe₃O₄ nanoparticles are made mixing Fe(acac)₃ in phenyl ether. 1,2-hexadecanediol, oleic acid, and oleylamine under nitrogen then heating to 260° C. and refluxed for 30 nuns. After cooling to room temperature, black colored magnetite crystals are isolated by adding an excess amount of ethanol followed by centrifugation (Jun, Y. H., Y M; Choi, J S; Sub, J S; Cheon, J, Nanoscale Size Effect of Magnetic Nanocrystals and Their Utilization for Cancer Diagnosis via Magnetic Resonance Imaging. JACS, 2005. 127: p. 5732-5733). To obtain larger sized nanocrystals, seed-mediated growth is used where smaller 4-nm Fe₃O₄ nanoparticles are mixed with additional precursor materials as previously described. By the controlling the quantity of nanoparticle seeds, Fe₃O₄ nanoparticles with various sizes can be formed. For example 62 mg of Fe₃O₄ seed nanoparticles leads to 12-nm nanoparticles, while changing the mass of seeds into 15 mg leads to 16-nm Fe₃O₄ nanoparticles (Sun, S. Z., H, Size-Controlled Synthesis of Magnetite Nanoparticles. JACS, 2002. 124: p. 8204-8205). To obtain bimetallic iron oxide particles such as CoFe2O₄ the aforementioned protocol is followed except that metal precursor of Co, Mn or Ni is added, at the half equivalence of iron precursor (Fe(acac)₃) used.

### 5.3 Nanoparticle Functionalization

Nanoparticles can be functionalized using methods known in the art to endow them with certain properties, such as biocompatibility, hydrophilicity (to enable aqueous suspension), specific cellular affinity and other functionalities that can enhance in situ effects.

For example, coating or encapsulation of magnetic nanoparticles can be used to render them biocompatible (or improve biocompatibility). Materials and methods for coating magnetic nanoparticles are known in the art. For example, U.S. Pat. No. 7,074,175 (entitled “Thermotherapy is targeted delivery of nanoscale magnetic particles,” Handy et al., Jul. 11, 2006) at cols. 11-12, discloses suitable materials and methods for coating magnetic nanoparticles. Suitable materials for the coating include synthetic and biological polymers, copolymers and polymer blends, and inorganic materials. Polymer materials may include various combinations of polymers of acrylates, siloxanes, styrenes, acetates, alkylene glycols, alkylenes, alkylene oxides, parylenes, lactic acid, and glycolic acid. Further suitable coating materials include a hydrogel polymer, a histidine-containing polymer, and a combination of a hydrogel polymer and a histidine-containing polymer.

Coating materials may include combinations of biological materials such as a polysaccharide, a polyaminoacid, a protein, a lipid, a glycerol, and a fatty acid. Other biological materials for use as a coating material may be a heparin, heparin sulfate, chondroitin sulfite, chitosan, cellulose, dextran, alginate, starch, carbohydrate, and glycosaminoglycan. Proteins may include an extracellular matrix protein, proteoglycan, glycoprotein, albumin, peptide, and gelatin. These materials may also be used in combination with any suitable synthetic polymer material.

Inorganic coating materials may include any combination of a metal, a metal alloy, and a ceramic. Examples of ceramic materials may include a hydroxyapatite, silicon carbide, carboxylate, sultanate, phosphate, ferrite, phosphonate, and oxides of Group IV elements of the Periodic Table of Elements. These materials may form a composite coating that also contains any biological or synthetic polymer. Where the magnetic particle is formed from a magnetic material that is biocompatible, the surface of the particle itself operates as the biocompatible coating.

The coating material may also serve to facilitate transport of the nanoparticle into a cell, a process known as transfection. Such art-known coating materials, known as transfection agents, include vectors, prions, polyaminoacids, cationic liposomes, amphiphiles, and non-liposomal lipids or any combination thereof. A suitable vector may be a plasmid, a virus, a phage, a viron, a viral coat. The nanoparticle coating may be a composite of any combination of transfection agent with organic and inorganic materials, such that the particular combination may be tailored for a particular type of a diseased cell and a specific location in a tissue or organ.

Hydrophilic, biocompatible, functional nanoparticles for use in the methods of the invention can be made from hydrophobic nanoparticles using methods known in the art. For example, hydrophobic nanoparticles can be made suspendable in aqueous solutions by introducing ionic or polar groups on the nanoparticle surface using methods known in the art. Depending on the starting surface properties of the ‘as-synthesized’ panicles, this can be accomplished, for example, by linking molecules to the nanoparticle surface through art-known chemisorption (e.g. thiol-metal interactions), to reactive groups on the particle surface that may have been introduced in the synthesis process through covalent bonds through, through coordination bonds, ionic bonds, pi-bonds, or hydrophobic interactions. Typically, a molecule can be ‘multi-functional,’ with one portion of the molecule exhibiting, affinity to the particle or particle surface groups and another portion of the molecule having: characteristics that would make the conjugate hydrophilic. This same portion of the molecule or another portion could also render the conjugate biocompatible, a typical example would be a molecule terminated with a polyethylene glycol (PEG) chain. This same portion of the molecule or another portion could also introduce additional functionality on the conjugate surface for conjugating additional materials onto the nanoparticle.

Nanoparticle functionalization strategies are well known to the skilled artisan. See, for example, Monodisperse magnetic nanoparticles for biomedical applications, Xu et al., Polymer International 56 (7), 821-826 (DOI: 10.1002/pi.2251) for a review of routine nanoparticle functionalization strategies.

In a specific embodiment, phospholipids can be used to encapsulate as-synthesized hydrophobic nanoparticles and render them suspendable in aqueous solutions. Depending on the phospholipid's properties, this strategy can also render the particles amenable to further functionalization (see, e.g., Benoit Dubertret, P. S., David Norris, Vincent Noireaux, Ali H. Brivanlou, and Albert Libchaber, In Vivo Imaging of Quantum Dots Encapsulated in Phospholipid Micelles, Science 2002. 298(5599): p. 1759-1762).

### 5.4 Nanoparticle Functionalization Using Targeting Ligands

To ensure that the nanoparticle selectively attaches to the target cells or tissues, in certain embodiments, one or more targeting ligand can be conjugated to, or combined with, the nanoparticle. Such targeting ligands are well known in the art. For example, U.S. Pat. No. 7,074,175 (entitled “Thermotherapy via targeted delivery of nanoscale magnetic particles,” Handy et al., Jul. 11, 2006) at cols. 12-15, discloses targeting ligands useful in targeting markers on target cells or tissues.

The association of one or more targeting ligands with the nanoparticle allows for targeting of cell- or tissue-specific markers on the target cell or tissue. The term ligand relates to compounds that may target molecules including, for example, proteins, peptides, antibodies, antibody fragments, saccharides, carbohydrates, glycans, cytokines, chemokines, nucleotides, lectins, lipids, receptors, steroids, neurotransmitters, Cluster Designation/Differentiation (CD) markers, and imprinted polymers and the like. The preferred protein ligands include, for example, cell surface proteins, membrane proteins, proteoglycans, glycoproteins, peptides and the like. The preferred nucleotide ligands include, for example, complete nucleotides, complimentary nucleotides, and nucleotide fragments. The preferred lipid ligands include, for example phospholipids, glycolipids, and the like. The ligand may be covalently or non-covalently? bonded to, or physically interacted with, the magnetic particle or the coating. The ligand may be bound covalently, non-covalently or by physical interaction directly to an uncoated portion of the magnetic particle. The ligand may be hound covalently, non-covalently or by physical interaction directly to an uncoated portion of the magnetic particle and partially covered by the coating. The ligand may be bound covalently, non-covalently or by physical interaction to a coated portion of the bioprobe. The ligand may be intercalated to the coated portion of the bioprobe.

Antibodies can be attached to a nanoparticle for introducing specific cellular and tissue targeting. Any art-known antibody or antibody derivative, full length antibodies and antibody fragments, that contains an antigen-specific binding site can be used. Antibodies or antibody derivatives that have naturally existing affinity or with synthetically derived affinity can be used. Antibodies (or fragments or derivatives thereof) that can be conjugated to nanoparticles include, but are not limited to, polyclonal antibodies, monoclonal antibodies, chimeric antibodies, humanized antibodies, recombinant antibodies, bispecific antibodies, and immunologically active fragments of immunoglobulin molecules such as scFv. F(ab), dsFV and F(ab′)2 fragments, which can be generated by treating the antibody with an enzyme such as pepsin or papain. Methods for generating and expressing immunologically active fragments of antibodies are well known in the art (see, e.g., U.S. Pat. No. 5,648,237). Other fragments include recombinant single chain antibody fragments, peptides, and the like.

Bispecific antibodies are non-natural antibodies that bind two different epitopes that are typically chosen on two different antigens. Characteristics and design of bispecific antibodies are well known in the art. A bispecific antibody is typically comprised of two different fragment antigen binding regions (Fabs). A bispecific antibody may be formed by cleaving an antibody into two halves by cleaving the disulfide bonds in the Fc region only. Two antibody halves with different Fab regions are then combined to form a bispecific antibody with the typical “Y” antibody structure. One or more antibody-based ligands can be conjugated to the nanoparticle. Antibodies of virtually any origin can be used as ligands, provided that they bind the target marker on the cell or tissue of interest, although human, chimeric % and humanized antibodies may aid in avoiding a human patient's immunogenic response.

Nanoparticles that are conjugated to antibodies or other targeting molecules or ligands are well known in the art and can be synthesized using routine methods. For example, nanoparticles conjugated to antibodies and used for targeting to a desired tissue are described in U.S. Pat. No. 6,165,440 (entitled “Radiation and nanoparticles for enhancement of drug delivery in solid tumors,” Esenaliev, Dec. 26, 2000). U.S. Pat. No. 6,165,440 discloses nanoparticles conjugated to antibodies and their uses in targeting and treating solid tumors with thermotherapy induced by optical pulsed radiation (in the 0.2 um to 2 um spectral range) and with cavitation induced by ultrasonic radiation (in the frequency range from 20 to 500 kHz).

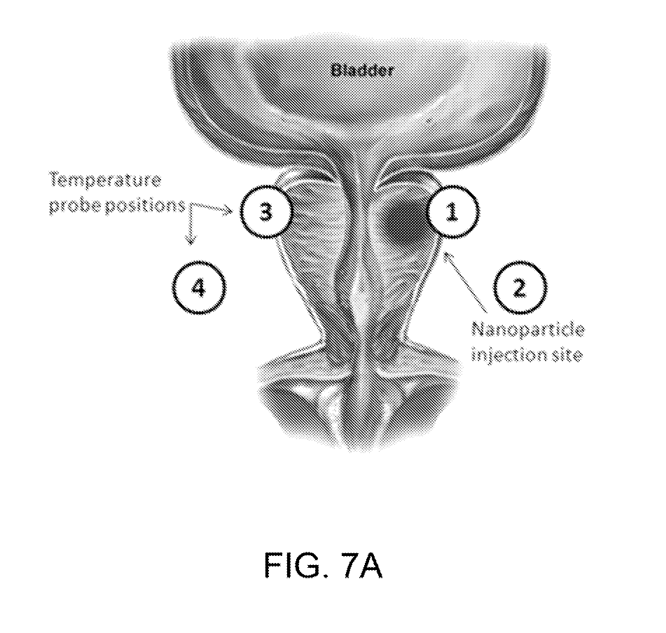

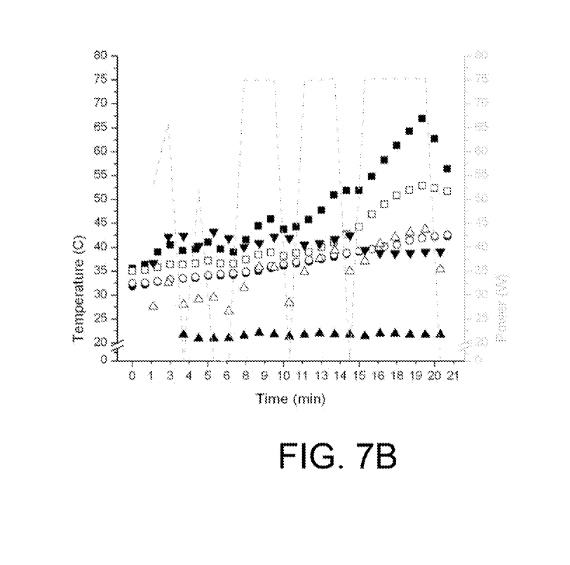

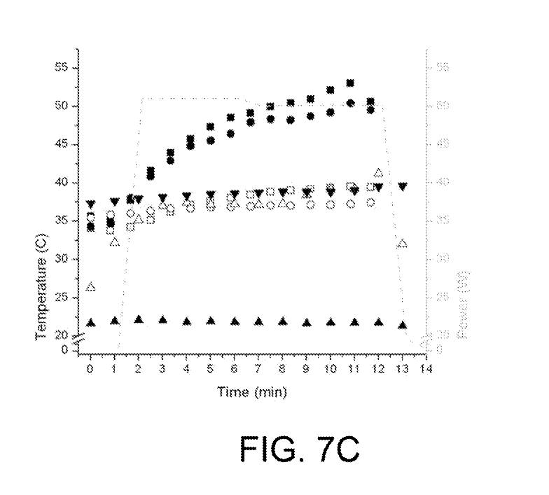

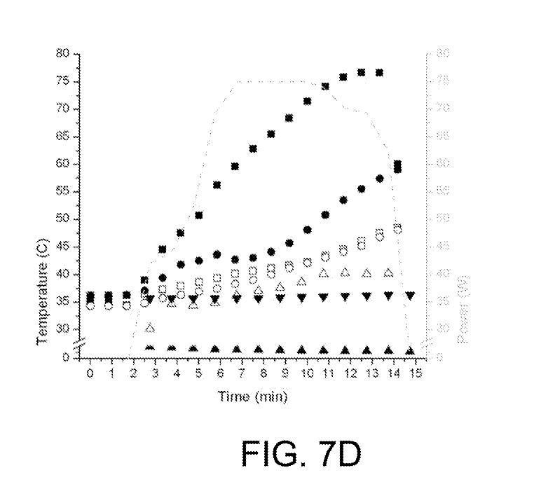

U.S. Pat. No. 7,074,175 (entitled “Thermotherapy via targeted delivery of nanoscale magnetic particles” Handy et al., Jul. 11, 2006) at cols. 12-15 and FIG. 7, discloses the characteristics of antibodies that can be attached as ligands to nanoparticles for targeting specific cells or tissues. For example, the antibody ligand may have a fragment crystallization (Fc) region and fragment antigen binding (Fab) regions. The Fab regions may be the antigen binding regions of the antibody that include a variable light region and a constant light region along with a variable heavy region and a constant heavy region. Biological activity of antibodies may be determined to a large extent by the Fc region of the antibody molecule. The Fc region may include complement activation constant heavy chains and macrophage binding, constant heavy chains. The Fc region and Fab regions may be connected by several disulfide linkages. Ligands that do not include the Fc region may be preferable in order to avoid immunogenic response. Examples of these ligands may include antibody fragments such as, fragment antigen binding fragments (Fabs), disulfide-stabilized variable region fragments (dsFVs), single chain variable region fragments (scFVs), recombinant single chain antibody fragments, and peptides.

An antigen binding fragment (Fab) may include a single Fab region of an antibody. A single Fab region may include a variable light and as constant light region bound to a variable heavy and a constant heavy region by a disulfide bond.

A disulfide-stabilized variable region fragment (dsFV) may include a variable heavy region and a variable light region of antibody joined by a disulfide bond. A leader sequence, which may be a peptide, may be linked to the variable light and variable heavy regions.

A single chain variable region fragment (scFV) may include a variable heavy region and variable light region of antibody joined by a linker peptide. A leader sequence may be linked to the variable heavy region.

Other targeting agents that can be attached to the nanoparticles include, but are not limited to, peptides and oligonucleotides, e.g. aptamers or spiegelmers, designed to target specific tissues or cells.

Molecules that have an affinity for, are taken up by, or are internalized or sequestered by a cell or tissue can also be used for conjugation to nanoparticles and targeting of that tissue. Such molecules, for example can have affinity for a cell-surface associated target, for a target associated with a cell uptake mechanism, or for an intracellular target. For example, iodine could be used for targeting of thyroid tissue. Folic acid could be used for targeting of cancer cells overexpressing the foliate receptor. Other small molecules suitable for use will be apparent to the skilled artisan.

Antibodies can be conjugated to the nanoparticles to target the nanoparticles to a specific tissue. For example, prostate-specific membrane antigen (PSMA) is a well-characterized type 2 integral membrane glycoprotein expressed in a highly restricted manner by prostate epithelial cells (He Liu, P. M., Sae Kim, Yan Xia, Ayyappan Rajasekaran Vincent Navarro, Beatrice Knudsen, Neil H. Bander. Monoclonal Antibodies to the Extracellular Domain of Prostate-specific Membrane Antigen Also React with Tumor Vascular Endothelium. Cancer Research, 1997. 57: p. 3629-3634). J591 is an anti-PSMA mAb that binds with 1-nM affinity to the extracellular domain of PSMA and is the subject of 11 clinical trials (see, He Liu, P. M., Sae Kim, Yan Xia, Ayyappan Rajasekaran, Vincent Navarro, Beatrice Knudsen, Neil H. Bander, Monoclonal Antibodies to the Extracellular Domain of Prostate-specific Membrane Antigen Also React with Tumor Vascular Endothelium. Cancer Research, 1997. 57: p. 3629-3634; Peter M. Smith-Jones, S. V., Stanley J. Goldsmith, Vincent Navarro, Catherine J. Hunter, Diego Bastidas, Neil H. Bander, In vitro characterization of radiolabeled monoclonal antibodies specific for the extracellular domain of prostate-specific membrane antigen. Cancer Research, 2000. 60: p. 5237-5243). An anti-PSMA mAb such as J591 is can be conjugated to the nanoparticles in order to target benign hyperplastic tissue.

Methods for conjugating ligands to nanoparticles are known in the art see, e.g., Bioconjugate Techniques, 2nd Edition, Greg T. Hermunson, Academic Press, Inc., 2008 (1202 pp); U.S. Pat. No. 7,074,175 entitled “Thermotherapy via targeted delivery of nanoscale magnetic particles,” Handy et al., Jul. 11, 2006, at cols. 12-13).

For example, a ligand may be covalently linked to the nanoparticle using a linker molecule. Linker molecules are well known in the art. A linker molecule is an agent that targets particular functional groups on the ligand and on the nanoparticle or the coating on the nanoparticle, and thus forms a covalent link between any two of these. Examples of functional groups used in linking reactions include amines, sulfhydryls, carbohydrates, carboxyls, hydroxyls and the like. The linking agent may be a homobifunctional or heterobifunctional crosslinking reagent, for example, carbodiimides, sulfo-NHS esters linkers and the like. The linking agent may also be an aldehyde crosslinking reagent such as glutaraldehyde. The linking agent may be chosen to link the ligand to the nanoparticle or the coating in a preferable orientation, specifically with the active region of the ligand available for targeting. Physical interaction does not require the linking molecule and the ligand be bound directly to the nanoparticle or to the coating, by non-covalent means such as, for example, absorption, adsorption, or intercalation.

### 5.5 In Vivo Nanoparticle Administration

According to the methods of the invention, microwave-active nanoparticles are introduced into target cells, tissues or organs of interest. Nanoparticles can be introduced into or loaded in or around, a target cell, tissue or organ in vivo using art-known methods and commercially available injection or administration systems.

The nanoparticles can be administered intravenously, intra-arterially, or locally to achieve specific loading in and around the target tissue.

Nanoparticles suspended in solutions suitable for in vivo administration can be injected systemically or locally or can be implanted or ‘seeded’ at specific sites within a target tissue using art-known methods. Systemic injection may require that the particles exhibit functionality that allows for specific targeting of prostate tissue cells in order to cause the particles to concentrate at the site to be treated. Local injection and seeding can be guided by Computed Tomography (see, e.g., Manfred Johannsen, U. G., Burghard Thiesen, Kasra Taymoorian, Chie Hee Cho, Norbert Waldofner, Regina Scholz, Andreas Jordan, Stefan A. Loening, Peter Wust, Thermotherapy of Prostate Cancer Using Magnetic Nanoparticles; Feasibility, Imaging, and Three-Dimensional Temperature Distribution. European Urology, 2007. 52: p. 1653-1662) and can also incorporate targeting, functionality so as to prevent removal or diffusion of the particles from the tissue through, e.g., the vasculature.

### 5.6 Microwaveable Materials

Microwave absorbing materials that generate dielectric and/or magnetic or polarization losses are known in the art and can be designed for use in the methods of the invention using routine methods known in the art. The mechanisms responsible for losses by materials may differ depending on the materials dimensions ranging from nanoscale versus micro and macro-scale (B. Lua, X. L. D., H. Huanga, X. F. Zhanga, X. G. Zhua, J. P. Leia, J. P. Suna, Microwave absorption properties of the core/shell-type iron and nickel nanoparticles. Journal of Magnetism and Magnetic Materials, 2008. 320: p. 1106-1111).

The electron transfer between Fe³⁺ and Fe²⁺ gives, rise to the ion jumps and relaxation Fe₃O₄ contributes a particular dielectric loss in iron oxide nanoparticles (B. Lua, X. L. D., H. Huanga, X. F. Zhanga, X. G. Zhua, J. P. Leia, J. P. Suna, Microwave absorption properties of the core/shell-type iron and nickel nanoparticles. Journal of Magnetism and Magnetic Materials, 2008. 320: p. 1106-1111).

Dielectric heating depends on a number of factors well known in the art, including the frequency of the microwave irradiation and the absorption properties of the dielectric at that frequency. All dielectric materials have characteristic absorption spectra (frequency versus heating ability). For example, in a conventional kitchen microwave oven, the microwave frequency (2.45 GHz) is good for heating water, but not good for heating other materials (for example, a cup that holds the water). By changing the frequency of the microwave emission, the cup can heated rather than the water (depending on the relative dielectric absorption characteristics of water and the cup). Thus, it is possible to heat materials in water without heating the water using dielectric heating. Once the material is heated, heat will transfer into adjacent water unless the heated material is covered with a heat-insulating layer.” Martin, M. Methods and Compositions for Directed Microwave Chemistry 2008. Mirari Biosciences, Inc.: USA.

The microwave emission(s) used in the methods of the invention can be in the range of 300 MHz to 300,000 MHz (approx. 3 m to 3 cm). Dielectric heating also occurs at longer (radio) wavelengths (up to 100 m), which can be alternatively used. Overall, dielectric heating frequencies span wavelengths of about 3 cm to 100 m, and in certain embodiments of the invention, dielectric heating frequencies are in this range. The exact frequency used can depend on the dielectric material to be heated. The dielectric can be more lossy than the solvent is at the chosen frequency, Martin, M. Method and Compositions for Directed Microwave Chemistry 2008, Mirari Biosciences, Inc.: USA.

In one embodiment, the frequencies used can be 0.915 GHz, 2.45 GHz, 5.85 GHz, and 22.125 GHz. In another embodiment, the frequencies used can be U.S. Government approved frequencies for use for industrial, scientific, and medical uses. Other frequencies may also be used provided that the emission within the microwave chamber is sufficiently shielded (to prevent interference with communications uses of microwaves) (Martin, M., Methods and Compositions for Directed Microwave Chemistry 2008, Mirari Biosciences, Inc.: USA).

In one embodiment, 0.915 GHz is used for aqueous applications because water is least susceptible to dielectric heating at this frequency (see Martin, M., Methods and Compositions for Directed Microwave Chemistry 2008, Mirari Biosciences, Inc.: USA).

Relative Loss Factors for distilled H₂O—Susceptibility to Microwave Heating

| 0.915 GHz | 2.450 GHz | 5.800 GHz |
|---|---|---|
| 1.41 | 11.3 | 4.3 |

The parameter that describes the ability of a dielectric material to convert electromagnetic, energy into other forms of energy (heat) is the dissipation factor or loss tangent (Tan δ). For every material, Tan δ is frequency dependent. Materials that have much higher values of Thud than the chosen solvent (at a given frequency), are attractive for this invention. The frequency can be chosen to optimize the ratio: Tan δ_{dielectric}/J Tan δₛₒₗᵥₑₙₜ. Thus, in a preferred embodiment, the microwave frequency and the absorbing characteristics of the dielectric (desired high absorbing) and solvent (desired low absorbing) are optimized (Martin. M., Methods and Compositions for Directed Microwave Chemistry 2008, Mirari Biosciences, USA).

For aqueous reactions it is preferable to use dielectric materials that have higher loss tangents than the solvent (if catalysis is desired). A list of exemplary materials that have higher values of Tan δ than water (as a solvent) is shown below. These materials, as well as others well known in the art that have higher loss tangents than the solvent can be used in the invention (Martin, M., Methods and Compositions for Directed Microwave Chemistry 2008, Mirari Biosciences, Inc.: USA).

| Frequency | Tanδ (water) | Tanδ (dielectric) |
|---|---|---|
| 3 GHz | 1570 | ethlylene glycol 10,000 |

(see, Martin, M., Methods and Compositions for Directed Microwave Chemistry 2008, Mirari Biosciences, Inc.: USA).

Effect of microwave heating on temperature of solids −1 min heating:

| | | |
|---|---|---|
| Water | 560 W, 2.45 GHz oven | 81 C. |
| Carbon: | 500 W, 2.45 GHz oven | 1283 C. |
| Nickel: | 500 W, 2.45 GHz | 384 C. |
| Copper oxide: | 500 W, 2.45 GHz | 701 C. (0.5 min heating) |

(see, Martin, M., Methods and Compositions for Directed Microwave Chemistry 2008, Mirari Biosciences, Inc.: USA).

| Material | Tanδ 915 MHz | Tanδ 2450 MHz |
|---|---|---|
| barium titanate | 0.20 | 0.30 |
| clay (20% water) | 0.47 | 0.27 |
| manganese oxide | 0.09 | 0.17 |
| Water | 0.043 | 0.12 |

(see, Martin, M., Methods and Compositions for Directed Microwave Chemistry 2008, Mirari Biosciences, Inc.: USA).

One material with a high dielectric constant well known in the art is barium titanate (BaTiO₃). The dielectric constant is 200-16,000 (compared with 80 for water). Barium titanate or other materials known to have high dielectric constants can be formed into films (Ewart et al., U.S. Pat. No. 5,922,537) and used in the methods of the invention. Moreover, in addition to barium titanate, methods are well known for forming thin and thick films of other ferroelectric materials at low temperature. Known high dielectric constant inorganic titanates, niobates, and ferroelectric polymers can be formed by many processes including low temperature chemical vapor deposition, laser photo-ablation deposition, sol-gel processes. RF magnetron sputtering, screen printing and firing, (in the case of the polymer) spin coating, and other methods (Yang. P. et al. (1998) Science 282, 2244). Natural clay can also be used as a moldable dielectric (see tables above).

In another embodiment, a 1:1 w/w mixture of alummamagnetite (Al₂O₃—Fe₃O₄) can be used as a dielectric support that heats strongly (Bram, G., Loupy, A., Majdoub, M., and Petit, A. (1991) Chem. Ind. 396). Clay differentiates itself from water as a microwave absorber at 915 MHz much more than at 2450 MHz (see table above) (Martin, M., Methods and Compositions for Directed Microwave Chemistry 2008, Mirari Biosciences, Inc.: USA).

Additional dielectric materials can be identified using conventional methods by screening dielectrics for their ability to heat substantially faster than solvents such as water during microwave irradiation. Class I dielectrics (dielectric constants typically less than 150) and Class II dielectrics (dielectric constants typically in the range of 600-18,000) can be used (Technical brochure, Novacap, Inc., Valencia Calif.). Other suitable materials include organic polymers, aluminum-epoxy composites, and silicon oxides. The microwave frequency can be varied as well. This simple screening procedure would yield conditions (frequency and material) that would direct heating toward the dielectric material without substantially heating water. Combinatorial synthesis of materials to discover those with attractive qualities such as unique dielectric properties is well known in the art (see, e.g., Schultz et al., U.S. Pat. No. 5,985,356).

Other materials that heat substantially under RF irradiation and that can be used in the methods of the invention include ferrites and ferroelectrics. Other types of materials that are well known to heat dramatically under microwave irradiation are various ceramics; oxides (Al₂O₃, for example), non-oxides (CrB and Fe₂B, for example), and composites (SiC/SiO₂, for example). Numerous materials are processed (sintered, etc.) by exploiting their microwave heating characteristics. (National Academy of Sciences USA, 1994).

Composite materials can be heated by microwaves and used in the methods of the invention. For example, materials that are normally transparent to microwaves can be heated by adding polar liquids or conducting particles. Refractory oxides such as alumina, mullite, zircon. MgO, or Si₃N₄ have been made to couple effectively with microwaves by the addition of electroconductive particles of 35 SiC, Si, Mg, FeSi, and Cr₂O₃. Oxides of Al₂O 3, SiO₂, and MgO have been effectively heated by the addition of lossy materials such as Fe₃O₄ MnO₂, NiO, and calcium aluminate. Mixtures of conducting powders, such as Nb, TaC, SiC, MoSi₂, Cu, and Fe, and insulators such as ZrO₂, Y₂O₃ and Al₂O₃, have coupled well with microwaves. Various materials in solution (zirconium oxynitrate, aluminum nitrate, and yttrium nitrate) that are good couplers have also been added to enhance microwave absorption of powdered insulating oxides.

Addition of conductive materials in various shapes including powder, flake, sphere, needle, chip, or fiber, can be used to cause the heating, of low loss materials. For example carbon black or metal pieces with sizes ranging from 0.1-100 um can be used to increase the heating properties when used as inclusions. The nature and concentration of such materials can be optimized using routine methods.

Microwave absorbing materials based on conducting polymers are known in the art and could also be used in the methods of the invention (see, e.g. Laurent Olmedo, P. H., Franck Jousse, Microwave Absorbing Materials Based on Conducting Polymers. Advanced Materials, 1993, 5(5)).

### 5.7 Microwave Radiation for Nanoparticle-Mediated Thermotherapy

Microwave irradiation for nanoparticle-mediated thermotherapy can be administered using art-known methods for non-nanoparticle mediated (“normal”) thermotherapy. In some embodiments, lower-than normal microwave power (versus that employed in normal thermotherapy) can be employed. In a specific embodiment, microwave irradiation at 300 MHz (3×10⁸ Hz) to 300 GHz (3×10¹¹ Hz) is administered. Routine methods known in the art can be used to select which microwave frequencies are suitable for a particular cell/tissue, taking into consideration, for example dielectric properties of both tissue and nanoparticles, known or observable interactions between tissue and nanoparticles in various in vivo and ex vivo tissue models, as well understanding the differential physics involved of each application.

Devices for administering thermotherapy to various target tissues and organs are known in the art and commercially available, e.g., TUMT devices. Such systems typically combine, in a single device, art-known components such as a source of microwave radiation, an electronic system for monitoring of the microwave radiation, and a system for delivery of the radiation to the tissue. Such components are also commercially available separately.

Several companies have developed FDA approved commercial transurethral microwave thermotherapy (TUMT) systems that operate either at 1296 MHz or 915 MHz and that combine a source of microwave radiation, an electronic system for monitoring of the microwave radiation, and a system for delivery of the radiation to the tissue. Nanoparticles specifically designed with high saturation magnetization values and with resonant frequencies tuned to 1296 MHz or 915 MHz can thus be used in conjunction with these systems, as shown in the table below;

| Manufacturer | Model | Freq (MHz) |
|---|---|---|
| Boston Scientific | Prolieve | 915 ± 5 |
| Thermatrx | TMX-2000 | 915 ± 1 |
| Urologix | Prostatron version 2.0 | 1296 |
| Urologix | Prostaron version 2.5 | 1296 |
| Urologix | Targis | 915 ± 13 |
| Urologix | CTC* | 915 ± 13 |
| Dornier | UroWave | 915 |
| Prostalund | CoreTherm | 915 |

Sources of radiating energy other than microwaves, moreover, can be used according to the methods of the invention including radiation spanning the entire electromagnetic spectrum. In certain embodiments, a plurality of radiating energy types can be used. A mixture of nanoparticles can be used that are tuned to the various radiating energy types in the plurality. Alternatively, a nanoparticle can be used that is tuned to a plurality of radiating energy types.

### 5.8 Methods of Treatment

A method for treating a cell or tissue of interest in as subject in need thereof is provided. The methods can comprise the steps of;

introducing microwave-active nanoparticles into the cell or tissue; and

applying a microwave field,

wherein:

the microwave-active nanoparticles react to microwave energy of the microwave field by releasing heat, and

the tissue is heated, thereby inducing hyperthermia or thermotherapy in the tissue.

In another embodiment, a method for treating cancerous tissue in a subject in need thereof is provided. The method can comprise the steps of;

introducing microwave-active nanoparticles into the cancerous tissue; and

applying a microwave field,

wherein:

the microwave-active nanoparticles react to microwave energy of the microwave field by releasing heat, and

the cancerous tissue is heated, thereby inducing hyperthermia in the cancerous tissue.

In one embodiment, the subject is an animal. In another embodiment, the animal is a human.

In another embodiment, the tissue is selected from the group consisting of prostate tissue, tumor tissue (e.g., benign or cancerous), solid cancer tissue, non-solid cancer tissue (e.g., leukemic, bone marrow cancer, or lymphogenic cancer tissue), bladder tissue, uterine tissue, uterine fibroid tissue.

In specific embodiments, methods for the treatment of prostate disorders such as BPH or prostate cancer using nanoparticle enhanced microwave thermotherapy are provided. The method for nanoparticle enhanced microwave thermotherapy for treatment of prostate disorders can be used as a minimally invasive outpatient therapy (e.g., performed in a doctor's office). Nanoparticles can be injected directly into the prostate either diffusely as a solution or placed as a seed aggregate using methods known in the art. Nanoparticle-mediated microwave treatment can also be guided with monoclonal antibodies targeting, e.g., BPH or cancer cells, either injected directly into the cell or tissue of interest (e.g., the prostate) or administered intravenously using art-known methods.

In one embodiment, microwave energy is delivered generally (non-locally) to die body (or a portion of the body) of a subject, with the nanoparticles in the targeted area being selectively activated by the microwaves. In another embodiment, microwave energy is delivered locally to a selected area or portion of the body of the subject. For example, in the case of a non-solid tumor, e.g., a bone marrow cancer, microwave irradiation can be focused on one portion of the circulatory system, e.g., a selected blood vessel through which blood flows.

Microwave energy for nanoparticle activation in the prostate can be delivered locally via transurethral, transrectal or transcutaneous pathways, or can be applied directly via surgery (e.g., open surgery or other suitable surgical procedure known in the art).

Low energy microwave energy that does not affect local tissue and is sequestered by nanoparticle activation for target tissue destruction can result in target tissue destruction with minimal side effects. Efficient destruction of as target tissue (e.g., BPH cells, cancer cells, tumor cells) can be accomplished in a minimally invasive manner that requires minimal analgesia or anesthesia ranging from intravenous or oral sedation to local anesthetic infiltration to regional and general anesthesia.

The method for nanoparticle enhanced microwave thermotherapy is equally applicable to other tissue pathologies such as solid or non-solid tumors or cancers. The method can be easily adapted by the ordinarily skilled practitioner for use in the treatment of other tissue pathologies, including tumors and cancers, by targeting cells or tissues of interest (e.g., cancer or tumor cells) with functionalized nanoparticles.

In a specific embodiment, nanoparticle-enhance microwave thermotherapy can be used for the treatment of transition cell carcinoma (TCC), which affects endothelial cells in the urinary bladder. In this embodiment, nanoparticles targeting TCC cells are applied through transdermal administration to the affected area by filling the bladder with a nanoparticle suspension. Once the particles are associated with the TCC cells, the affected area is washed to remove nonspecifically bound particles and then a TUMT device can be used to deliver microwave radiation to the area, thus heating the particles associated with TCC cells and treating the cancer with hyperthermia or the

Other pathologies that can be treated include, but are not limited to, uterine fibroids and other tumor and cancers including for example uterine, breast, colon, lymphatic, lymphogenic, hone marrow and many others as long microwave energy field can be delivered to that area and nanoparticle enhancement can be applied. Additional means of in vivo administration includes, but is not limited to, subcutaneous and oral administration. Microwave administration can also be achieved by means other than transurethral methods, using methods well known in the art, including, but not limited to, transrectal and transcutaneous application.

A method for aggregating nanoparticles in a cell or tissue of interest in a subject in need thereof is also provided. The method can comprise the step of introducing nanoparticles into the cell or tissue, wherein the nanoparticles are functionalized with a functional coating. The method can further comprise the step of applying a source of radiating energy. The nanoparticles can be designed or tuned to interact with energy from a radiating energy source. In a specific embodiment, the nanoparticle is tuned to interact with microwaves such that the particle is more lossy in the presence of microwaves than the cells or tissue of interest are,

### 5.9 Treatment Systems

A system is provided for controlling effects of a field of microwave radiation in a cell or tissue of interest in a subject in need thereof. The system can comprise:

a source of microwave radiation;

an electronic system for monitoring of the microwave radiation;

a system for delivery of the microwave radiation to the cell or tissue; microwave-active nanoparticles that absorb the microwave radiation;

an injection or administration system for administration of the nanoparticles;

wherein;

the microwave-active nanoparticles react to microwave energy of the field of microwave radiation by releasing heat, and

the cell or tissue is heated, thereby inducing, hyperthermia or thermotherapy in the cell or tissue, and whereby the effects of the field of microwave radiation are controlled.

Sources of microwave radiation, electronic systems for monitoring microwave radiation, systems for delivery of microwave radiation to cells or tissues, and injection or administration system for administration of the nanoparticles are known in the art and commercially available.

Adverse or unwanted effects of microwave irradiation of cells or tissues are well known in the art, and can include, but are not limited to inadvertent destruction of adjacent tissue or cells resulting in unwanted complications such as stricture, fistula or other inadvertent results of unwanted local damage.

In one embodiment, adverse or unwanted effects are controlled by using the methods of the invention to alter hydration of a biological target. In another embodiment, the microwave field of radiation is altered by modifying antenna design. In another embodiment, the effects are controlled by using direct cooling or application of pressure to biological target, e.g., in designs for transurethral, transrectal and other natural orifice route of entry as well as transcutaneous, and other routes of open surgical access.

The following examples are offered by way of illustration and not b way of limitation.

### 6. EXAMPLES

### Nanoparticle-Mediated Microwave Treatment Methods

Microwave-active nanoparticles can be created for use in focused microwave thermotherapy inside the prostate with precision to the cellular level. An advantage of using nanoparticles with diameters ranging from 4-20 nm is that heat transfer is rapid to the surrounding tissue and reduces temperature gradients. Furthermore, this technique permits reduced microwave power below current treatment levels thereby minimizing the risk for side-effects while still allowing thr the localized deliver of effective thermal doses to targeted tissue.

### 6.1 Example 1

### Design, Synthesis and Characterization of Magnetic Nanoparticles Targeted to the Prostate

This section describes the design, synthesis and characterization of microwave-active magnetic nanoparticles that are targeted toward a prostate antigen.

The major parameters that can be optimized are:

The size and composition of the nanoparticle and microwave-induced heating capacity.

The capping chemistry.

The functionalization to couple the antibody.

6.1.1 Nanoparticle Synthesis

Rational syntheses are carried out to build a library of nanomaterials that can strongly absorb microwave radiation. A series of metal-doped magnetism-engineered iron oxide (MEIO) nanoparticles of spinet MFe₂O₄ where M is +2 cation of Mn, Fe, Co or Ni (Lee, J. H. Y M; Jun, J W; Jang, J T; Cheon, J, Artificially engineered magnetic nanoparticles for ultra-sensitive molecular imaging; Nature medicine. Nature Medicine, 2007. 13(1): p. 95-99) are investigated.

Particles are synthesized following published literature protocols. Briefly, 4-nm Fe₃O₄ nanoparticles are made mixing Fe(acac)₃ in phenyl ether, 1,2-hexadecanediol, oleic acid, and oleylamine under nitrogen then heating to 260° C. and refluxed for 30 mins. After cooling to room temperature, black colored magnetite crystals are isolated by adding an excess amount of ethanol followed by centrifugation (Jun, Y. H., Y M; Choi, J S; Sub, J S; Cheon, J, Nanoscale Size Effect of Magnetic Nanocrystals and Their Utilization for Cancer Diagnosis via Magnetic Resonance Imaging. JAGS, 2005. 127: p. 5732-5733). To obtain larger sized nanocrystals, seed mediated growth is used where smaller 4-nm Fe₃O₄ nanoparticles are mixed with additional precursor materials as previously described. By the controlling the quantity of nanoparticle seeds, Fe₃O₄ nanoparticles with various sizes can be formed. For example 62 mg of Fe₃O₄ seed nanoparticles leads to 12-nm nanoparticles, while changing the mass of seeds into 15 mg leads to 16-nm Fe₃O₄ nanoparticles (Sun, S. Z., H, Size-Controlled Synthesis of Magnetite Nanoparticles. JACS: 2002. 124: p. 8204-8205). To obtain bimetallic iron oxide particles such as CoFe₂O₄, the aforementioned protocol is followed except that metal precursor of Co, Mn or Ni is added, at the half equivalence of iron precursor (Fe(acac)₃) used.

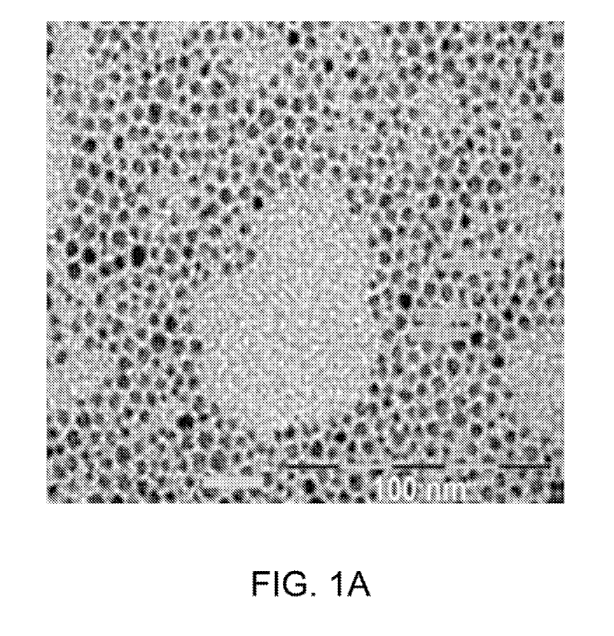

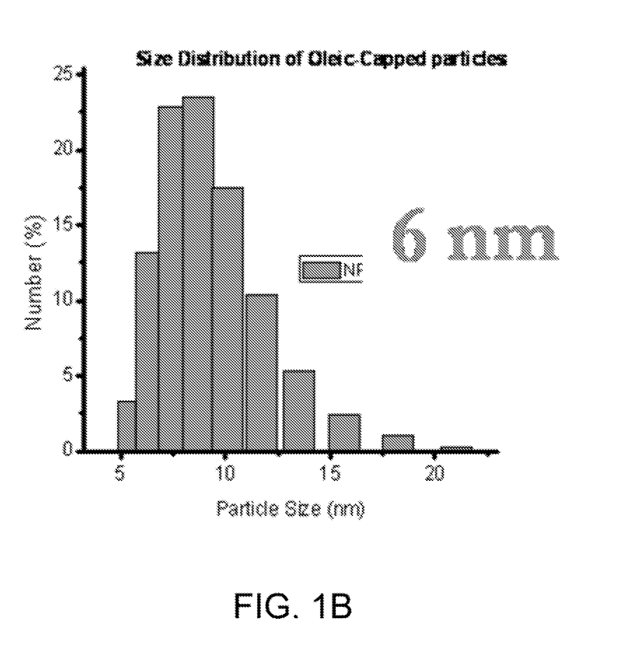

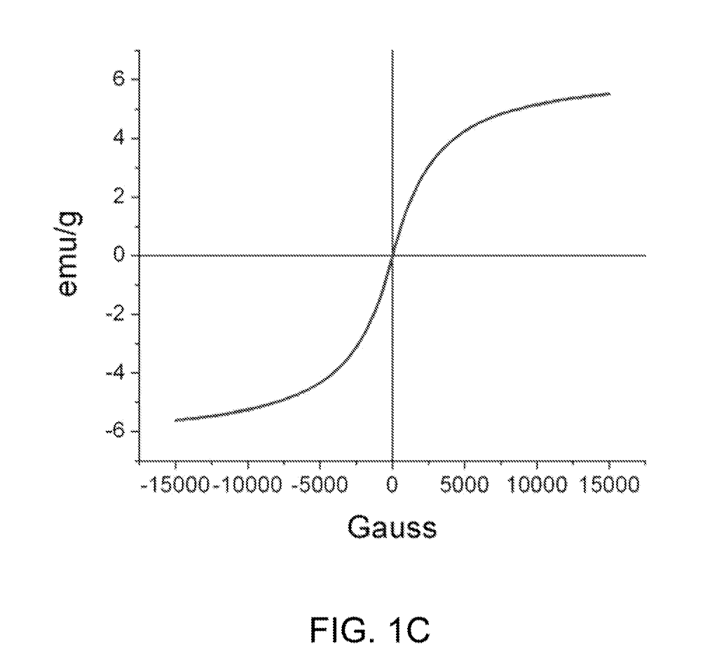

According to the above-described methods, superparamagnetic Fe₃O₄ nanoparticles ˜6 nm in diameter have been synthesized through thermal decomposition of Ferric salt under nitrogen using oleic acid as a surfactant. The resulting Fe₃O₄ particles were capped with oleic acid and exhibit a saturation magnetization of ˜6 emu/g (FIGS. 1A-C).

6.1.2 Nanoparticle Functionalization to Make Water Soluble Nanoparticles

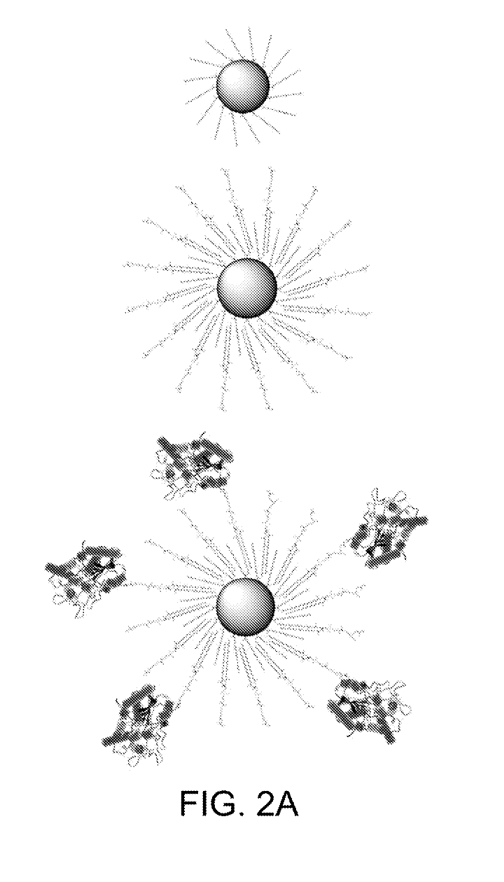

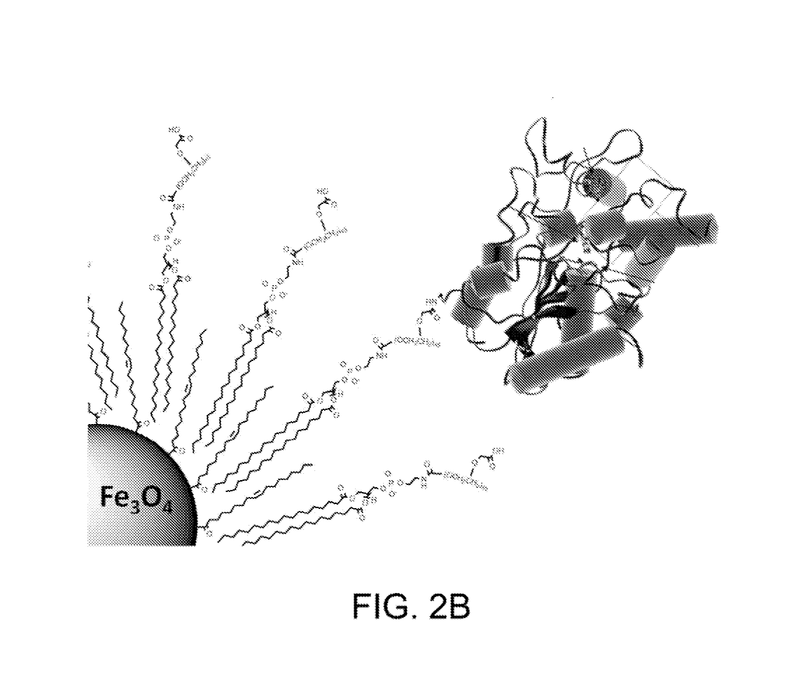

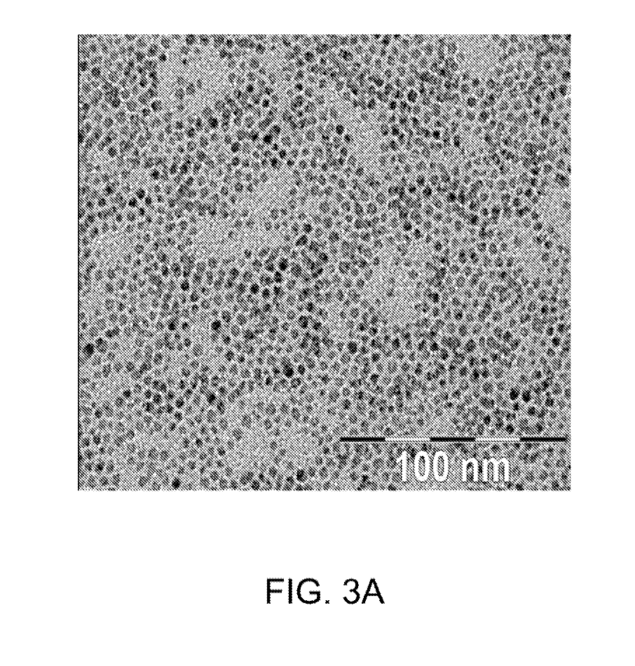

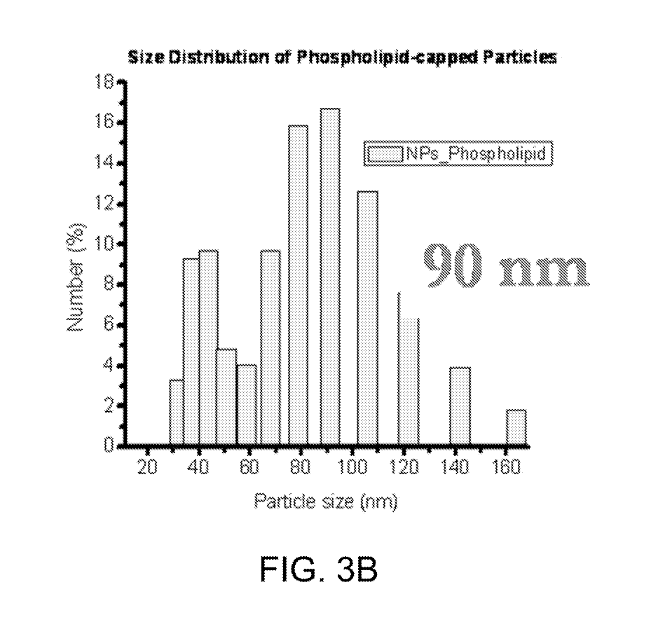

A nanoparticle can be functionalized to provide a functional cap that protects the particle and enhances its utility and makes it water soluble (FIGS. 2 A-B). Oleic acid-capped ‘as-synthesized’ nanoparticles can be functionalized following published methods (Benoit Dubertret, P. S., David J. Norris, Vincent Noireaux, Ali H. Brivanlou, and Albert Libchaber, In Vivo Imaging of Quantum Dots Encapsulated in Phospholipid Micelles. Science 2002. 298(5599): p. 1759-1762). Briefly. ‘as-synthesized’ nanoparticles in powder form are suspended in chloroform with carboxy-terminated PEG phospholipids (1,2-Distearoyl-sn-Glycero-3-Phosphoethanolamine-N-[Carboxy(Polyethylene Glycol)2000], Avanti Polar Lipids, Inc., Alabaster, Ala.). The chloroform is allowed to evaporate at room temperature and the residue is then be heated at 80° C. Next the residue is reconstituted in water and the suspension is spun at 500,000×g via ultracentrifugation where micelles containing particles will form a pellet while the empty micelles will stay suspended. The supernatant is then discarded and the particle-micelles are resuspended in water.

6.1.3 Microwave-Induced Heating Capacity in a Tissue-Equivalent Phantom

Guy has derived formulations for phantom models specifically designed for preclinical studies of the effects of microwave radiation on human tissue (Guy, A. W., Analyses of Electromagnetic Fields induced in Biological Tissues by Thermographic Studies on Equivalent Phantom Models, IEEE Transactions on Microwave Theory and Techniques, Volume 19, issue 2, February 1968: 205-214). Based on Guy's methods, Chou et al. published formulas for preparing tissue-equivalent phantom models with similar dielectric properties of muscle tissue at specific frequencies, including 915 MHz (Chou C K, C. G., Guy A W, Luk K H, Formulas for Preparing Phantom Muscle Tissue at Various Radiofrequencies. Bioelectromagnetics, 1984. 5: p. 435-441). This phantom is composed of polyethylene powder, water, sodium chloride and TX-151, a gelling agent (Oil center Research International, Lafayette, La.).

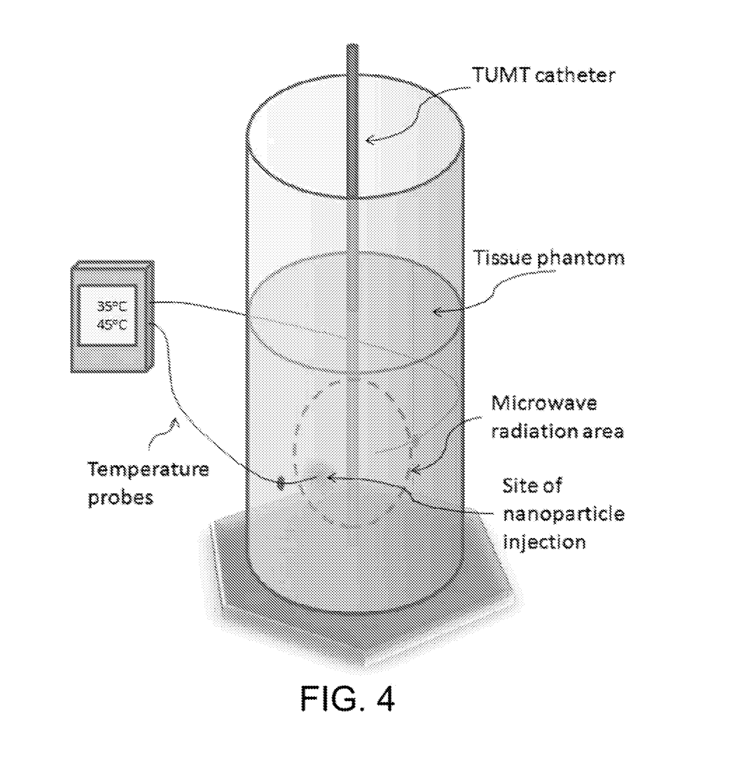

Following the published protocol of Chou et al., a phantom can be produced which, at room temperature (22° C.), simulates real tissue at 37° C. The mixture is prepared as described in Chou et al. and then poured into a cylindrical cast made of transparent plastic with a diameter of 10 cm and a length of 30 cm (FIG. 4). A Urologix Targis TUMT catheter antenna is positioned in the center of the mold and the phantom is then left to solidify at room temperature. After the phantom is solidified, a suspension of PEGylated nanoparticles in 0.5 cc of water is injected 2 cm away from the Targis antenna through a small hole in the phantom mold.

Next a 0.4 mm-diameter fiber-optic temperature probe (T1 Fiber Optic Probe, Neoptix, Québec, Canada) is inserted through the same hole and positioned such that the tip of the probe is within the nanoparticle volume. Another fiber-optic probe is inserted through another small hole in the phantom mold and is positioned at a position directly opposite of the first probe and also 2 cm away from the antenna. The temperature probes are then connected to a temperature sensor (Reflex Signal Conditioner, Neoptix, Quebec, Canada) that allows real-time temperature measurements during microwave application and the Targis catheter is connected to the Targis microwave generator and control system.

During experimentation, microwave energy is applied following, a period of 30 minutes following clinical protocol and temperature measurements are recorded and stored in a laptop computer connected to the Reflex sensor. The temperature measurements from the two probes is plotted, over time using software provided by Neoptix and the heating characteristics at site of nanoparticle injection is analyzed.

The nanoparticles will cause enhanced heating at the site of injection and will therefore allow for reduced microwave energy as compared to normal clinical procedure while still delivering sufficient thermal doses in the nanoparticle volume. Thus subsequent experiments can be conducted in which the treatment time is reduced at normal microwave power and the microwave power is reduced for normal treatment times. Such experiments can also be conducted by varying the nanoparticle concentration while administering a constant volume of 0.5 cc. By analyzing the resulting temperature measurements within the nanoparticle volume during modified procedures, data for the capabilities of this method and an optimal treatment protocol can be assessed.

### 6.2 Example 2

### Nanoparticle Functionalization to Make Functional Nanoparticles

The carboxy-terminated functionalized nanoparticles resulting from the phospholipid functionalization described above can be further modified by covalently attaching J591 antibody. In both cases, the carboxyl groups on the nanoparticles are converted to primary-amine-reactive NHS-esters using EDC (1-ethyl-3-[3-dimethylaminopropyl]carbodiimide hydrochloride), and sun-NHS (N-Hydroxysulfosuccinimide) (Pierce Biotechnology, Rockford, Ill. USA) following the manufactures protocols.

Next, J591 at 10-fold concentrations Over nanoparticle concentrations is added to the NHS-ester-modified particles suspended in phosphate buffered saline. The mixture is allowed to react for 2 hours during which time the NHS-esters will react with primary amines on the proteins forming a stable amide bond. Excess, unconjugated protein is then separated from the nanoparticle-conjugates through size exclusion chromatography using on an FPLC system (Superdex 200 size exclusion column on an AKTA Explorer FPLC, Amersham Biosciences, Piscataway, N.J. USA).

The nanoparticle-conjugated antibody activity is then assessed through Surface Plasmon Resonance (SR 7000 SPR Refractometer, Reichert, Depew, N.Y. USA) where the conjugate's binding affinity to PSMA is verified and compared to that of free J591. In a typical protocol, PSMA is immobilized on an SPR chip following the manufacturer's protocols. Next, J591 antibody is allowed to flow over the chip surface where it binds to immobilized PSMA. The binding event is recorded by the SPR system that measures chanced in the index of refraction at the chip surface caused by interactions between binding molecules and surface plasmon derived, from the chip. The antibody is then knocked off under basic conditions and the procedure is repeated for varying concentrations of antibody. The gathered binding data can then be used to determine the kinetic characteristics of the J591-PSMA interaction. This procedure can also be conducted with J591-functionalized nanoparticles and in this manner, the binding affinity of the nanoparticle conjugates can be compared to that of free J591.

### Example 3

### In Vitro Cell Culture Studies

In vitro studies can be conducted on prostate epithelial cells in order to assess the targeting capability of the nanoparticle conjugates as well as nanoparticle-directed polymer formation. For these experiments, functionalized nanoparticles are stained with the hydrophobic fluorescent dye acridine orange which loads in the hydrophobic region of the phospholipids that encapsulate the nanoparticles and allows for observation of nanoparticle aggregates through fluorescence confocal microscopy,

PSMA expressing, immortalized benign prostate hyperplasia endothelial cells (BPH-1, German Collection of Microorganisms and Cell Cultures, Braunschweig, Germany) and PSMA non-expressing prostate endothelial carcinoma cells (PC-3, American Type Culture Collection, Rockville, Md., USA) are cultured in 75 cm² ‘T-flasks’ in their respective media according to the manufacturers protocols. The cells are then sub-cultured during mid-log-phase growth; 5000 cells in 1 mL of the culture medium are transferred onto 35 mm cell culture dishes (Corning). Cells are allowed to grow for 12 hours at 37° C. and 5% CO2. After 12 hours, various concentrations of fluorescently labeled nanoparticle conjugates suspended in cell culture media are added to each cell line and the cells are then allowed to further incubate at 37° C. and 5% CO₂ for various time intervals.

Afterward, the cells are washed with fresh culture media in order to remove any particles that are not specifically associated with cells. A tier washing, 1 mL of culture media are added to each dish and the cells are stained with a red fluorescent membrane dye (FM464, Invitrogen) for 10 minutes. The cells are then be washed again and left with 1 mL of fresh culture media for imaging under confocal microscopy (Leica TCS SP2) using an immersion lens.

Acridine orange-labeled phospholipid encapsulated Fe₃O₄ nanoparticles have been prepared that are conjugated with single chain fragments of the variable region (scFv) of an antibody targeting the A33 cell surface glycoprotein and that is expressed human colon endothelial cell carcinomas.

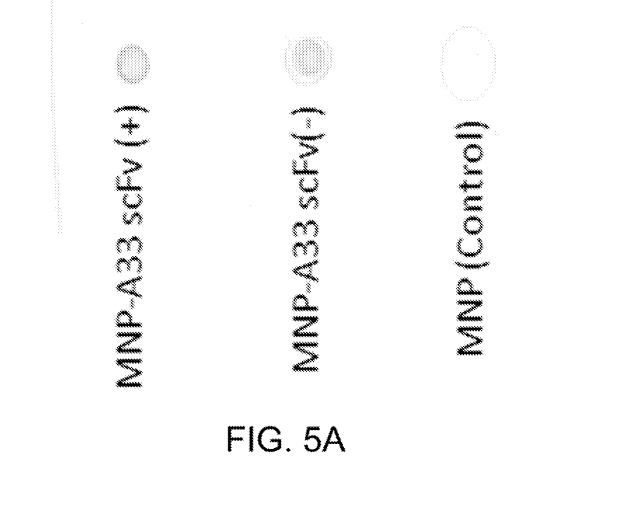

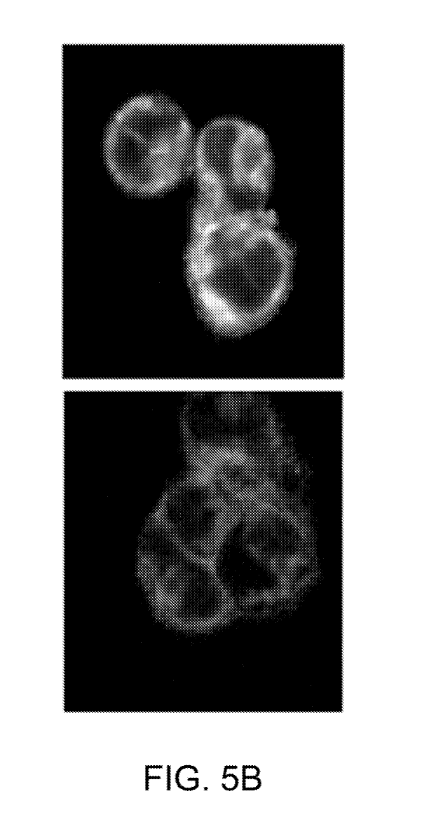

FIG. 5A shows a dot blot demonstrating the presence of antibody on the nanoparticles.

FIG. 5B shows cell culture experiments targeting A33 antigen expressing SW1222 colon cancer cells (top) and not targeting A33 antigen non-expressing HT29 cells (bottom).

Through confocal microscopy (FIG. 5B), specific affinity of the antibody conjugated nanoparticles (scFv(+)) to the A33 expressing SW1222 cell line was observed. This affinity was significantly decreased for nanoparticles conjugated to a modified version of the scFv without A33 affinity (scFv(−)). In this manner, samples containing J591 conjugated nanoparticles can be analyzed for specific targeting capabilities to BPH-1 cells.

### 6.4 Example 4

### Nanoparticle Synthesis

6.4.1 Nanoparticle Growth

This example demonstrates the successful synthesis and functionalization of magnetic nanoparticles (MNPs).

Nanoparticles were synthesized following Jun et. al (2005, Nanoscale Size Effect of Magnetic Nanocrystals and Their Utilization for Cancer Diagnosis via Magnetic Resonance Imaging. JACS, 2005. 127: p. 5732-5733). Briefly, 4-nm Fe₃O₄ nanoparticles were made mixing Fe(acac); in phenyl ether, 1,2-hexadecanediol, oleic acid, and oleylamine under nitrogen then heating to 260° C. and refluxed for 30 minutes. After cooling to room temperature, black colored magnetite crystals were isolated by adding an excess amount of ethanol followed by centrifugation. This results in monodispersed MNPs (FIGS. 1A-C) with an oleic acid coating.

6.4.2 Nanoparticle Coating

The platform functionalization strategy employed in this work followed Benoit Duhertret et al. (Benoit Dubertret, P. S., David J. Norris, Vincent Noireaux, Ali H. Brivanlou, and Albert Libchaber, In Vivo imaging of Quantum Dots Encapsulated in Phospholipid Micelles. Science 2002. 298(5599): p. 1759-1762) and utilized 1,2-Distearoyl-sn-Glycero-3-Phosphoethanolamine-N-[Carboxy(Polyethylene Glycol) 2000] (PL-PEG-COOH) (Avanti Polar Lipids, Alabaster, Ala. USA). These molecules consisted of phospholipids attached to 45-unit polyethylene glycol (PEG) with a carboxylic acid at its terminus that can be used for further chemical modification.

6.4.3 Nanoparticle Functionalization

Proteins were attached to MNP-PL-PEG-COOH by converting the carboxyl groups to primary amine-reactive NHS-esters using EDC (1-ethyl-3-[3-dimethylaminopropyl]carbodiimide hydrochloride), and sulfo-NHS (NHydroxysulfosuccinimide) (Pierce Biotechnology, Rockford, Ill. USA) following the manufactures protocols (FIGS. 2A-B).

Excess reagents including EDC/sulfo-NHS and excess and unconjugated protein were removed from MNP-conjugate solutions via size exclusion chromatography using Superdex 200 resin and an AKTA Explorer FPLC (GE Healthcare).

### 6.5 Example 5

### Antibody-Mediated Cell Targeting of Nanoparticles

Nanoparticles were functionalized with an antibody fragment to assess their targeting capabilities. Nanoparticles were functionalized with the humanized single-chain variable domain fragment antibody (scFv) derived from the monoclonal antibody A33 recognizes the A33 cell surface glycoprotein expressed in colorectal cancers.

The cell surface differentiation antigen (A33) of normal human gastrointestinal epithelium is expressed in 95% of primary and metastatic colorectal cancer cells, but is absent in most other normal tissues and tumor types.

After functionalization, characterization of A33scFv conjugation was verified via dot blot (FIG. 5A). MNP's were conjugated to A33scFv as well as a control A33scFv modified to not target the A33 antigen and functionalization assessed with Protein-L.

Cell targeting was verified using SW 1222 (A33 expressing cells) and HT29 (A33 non-expressing cells) (FIG. 5B).

### 6.6 Example 6

### Nanoparticle-Mediated Microwave Thermotherapy in Prostate

This example demonstrates the successful application of nanoparticle-mediated microwave thermotherapy in the prostate.

6.6.1 Introduction

More than half of the men in the United States between the ages of 60 and 70 and as many as 90 percent between the ages of 70 and 90 have symptoms of Benign Prostate Hyperplasia (BPH), also known as enlarged prostate, which obstructs the urethra.

Transurethral Microwave Thermotherapy (TENT) is a common treatment for BPH symptoms that consists of a catheter-based system containing a microwave antenna used to deliver microwave radiation from the urethra and into the prostate tissue. The device delivers microwave radiation to the prostate to achieve intraprostatic temperatures sufficient to result in tissue necrosis and the dilation of the prostatic urethra. Specific targeting of obstructive intraprostatic tissue is critical so as not to damage non-target areas such as the rectum, the urinary sphincters, and the penis. This limits the efficacy of TUMT devices. The use of MNPs was investigated for use in focused microwave thermotherapy inside the prostate with precision to the cellular level. This technique can permit reduced microwave power below current treatment levels thereby minimizing the risk for non-targeted heating while still allowing for the localized delivery of effective thermal doses to targeted tissue.

6.6.2 Ex Vivo TUMT in Bull Prostate

TUMT experiments were conducted using a Urologix TUMT device applied to a bull prostate ex vivo, 3 cc of phospholipid-PEG coated particles at 2 mg/mL were injected into one side of the bull prostate. Microwave power was applied up to 40 W during a period of ˜18 minutes and intraprostatic temperatures were monitored using fiber optic temperature probes (Reflex Signal Conditioner, Neoptix, Québec, Canada).

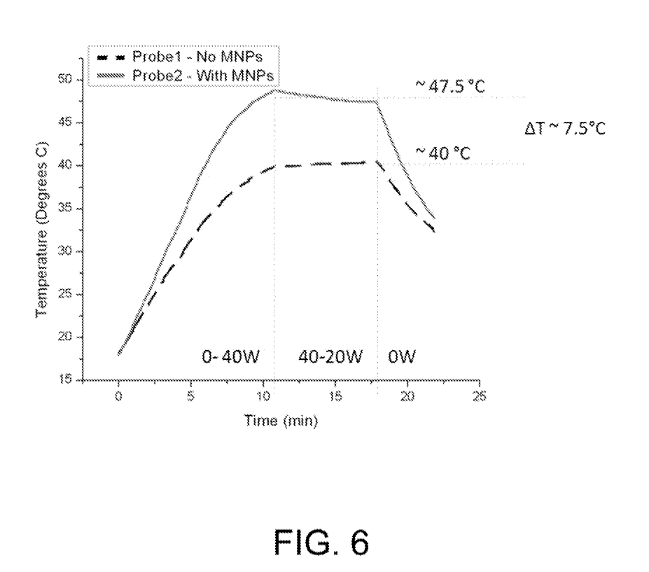

Probe 1 monitored an area with no MNPs Probe 2 monitored an area with MNPs. A ˜7.5° C. differential in temperature was achieved over a ˜8 min period (FIG. 6) which indicates that the method is feasible for use in vivo,

6.6.3 In Vivo TUMT in Canine Prostate

Previous studies (discussed above in Section 2) disclose the use nanoparticles for enhanced hyperthermia and thermotherapies with alternating magnetic fields in the kilo-hertz frequency range. These previous studies did not investigate, however, whether enhanced heating from nanoparticles can be achieved in vivo, i.e., whether microwave irradiation produces more heating in tissue targeted with nanoparticles than in tissue alone. They also did not investigate whether the heating differential achieved by microwaves is sufficient for therapeutic applications while maintaining a safe temperature in non-target tissue. The following experimental data obtained in vivo shows that there is indeed a therapeutically relevant heat differential achieved by microwaves irradiation while maintaining a safe temperature in non target tissue. Clinically, this shows that nanoparticles can be used to selectively target and heat selected tissues and/or cells to a higher temperature for destruction, while leaving neighboring cells and/or tissues viable, in the same organ (i.e., kill prostate cancer cells while leaving healthy ones without damage).

In vivo experiments were conducted on five, canines (beagles) between the ages of 5 and 6 years. Dogs were sedated while Urologix TUMT catheters were inserted into the urethra with the microwave antenna placed at the site of the prostate.

Prior to administration of Microwave power, 0.5 or 0.25 cc of phospholipid-PEG coated particles were injected into the right lobe of the canine prostate. Four fiber optic temperature probes (Reflex Signal Conditioner, Neoptix, Québec, Canada) were then inserted (FIG. 7A). Probes were positioned as follows (1) on the prostate at the site of injection, (2) lateral to the prostate on the side of injection, (3) on the prostate opposite of the side of injection, and (4) lateral to the prostate opposite of the side of injection.

Microwave power was then applied at varying intensities and intervals. FIGS. 7B-D summarize the temperature measurements during microwave administration for dogs 1-3, The temperatures labeled Coolant. MDS, and Rectal were those recorded by the Urologix machine. Coolant is the temperature of the coolant water that flows in the sheath of the catheter. MDS is the temperature of this water in the sheath at the site of the microwave antenna (i.e. in the urethra at the site of the prostate). Rectal is the temperature at the rectum.

In FIG. 7B, 0.5 cc of nanoparticle solution was administered to the first canine and the heating response of the particles due to varying microwave power was assessed. Prostatic temperatures at the site of injection immediately responded to power variations while opposite of the side of injection, only a gradual increase in temperature was observed. The temperature of untreated prostate tissue remained below 40° C. throughout the treatment, well below therapeutic levels of 50° C. while treated tissue reached over 65° C.

In FIG. 7C. 0.5 cc of nanoparticle solution were administered to the second canine and microwave power was applied at a constant level of 50 W. In this experiment, the nanoparticle injection diffused to both sides of the prostate after administration. This is reflected in the temperature measurements as seen in the temperature increase of both probes 1 and 3. Nonetheless, therapeutic temperatures of 50° C. were achieved at the prostate while lateral to the prostate the temperature remained below 40° C.

In FIG. 7D, 0.5 cc of nanoparticle solution were administered to the third canine and microwave power was applied at high intensity reaching 75 W. While at the prostate, opposite of the side of injection, therapeutic temperatures of 50° C. were achieved, the site of injection reached over 77° C. Furthermore, lateral to the prostate, the temperature remained below 45° C.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes,

The citation of any publication is for its disclosure prior to the filing date and should not be construed as an admission that the present invention is not entitled to antedate such publication by virtue of prior invention.

What is claimed is:

1. A method for treating a cell or tissue of interest in a subject in need thereof comprising the steps of
introducing microwave-active nanoparticles into the cell or tissue; and
applying a microwave field,
wherein:
the microwave-active nanoparticles react to microwave energy of the microwave field by releasing heat, and
the tissue is heated, thereby inducing hyperthermia or thermotherapy in the tissue.

2. The method of claim 1 wherein the cell or tissue is selected from the group consisting of prostate tissue, tumor tissue, solid cancer tissue, non-solid cancer tissue, leukemic cells, hone marrow cancer cells, lymphogenic cancer tissue, bladder tissue, uterine tissue, and uterine fibroid tissue.

3. The method of claim 1 wherein the step of applying the microwave field is selected from the group consisting of applying transurethrally, applying transrectally, applying transcutaneously, and applying directly via surgery.

4. The method of claim 1 wherein the nanoparticles are:
tuned to interact with microwaves such that the nanoparticles are more lossy in the presence of microwaves than the cells or tissue of interest are, and functionalized with a functional coating.

5. The method of claim 4 wherein the functional coating is a biocompatibility coating, an inorganic coating, or a hydrophilic coating.

6. The method of claim 4 wherein the functional coating comprises a targeting ligand and wherein the targeting ligand targets the cell or tissue of interest.

7. The method of claim 4 wherein the functional coating comprises a material that promotes nanoparticle aggregation within the cell or tissue of interest.

8. The method of claim 1 wherein the nanoparticles have diameters of 1-500 nm.

9. A method for treating cancerous tissue in a subject in need thereof comprising the steps of introducing microwave-active nanoparticles into the cancerous tissue; and
applying a microwave field,
wherein:
the microwave-active nanoparticles react to microwave, energy of the microwave field by releasing heat, and
the cancerous tissue is heated, thereby inducing hyperthermia in the cancerous tissue.

10. The method of claim 9 wherein the cell or tissue is selected from the group consisting of prostate tissue, tumor tissue, solid cancer tissue, non-solid cancer tissue, leukemic cells, hone marrow cancer cells, lymphogenic cancer tissue, bladder tissue, uterine tissue, uterine fibroid tissue.

11. The method of claim 9 wherein the step of applying the microwave field is selected from the group consisting of applying transurethrally, applying transrectally, applying transcutaneously, and applying directly via surgery.

12. The method of claim 9 wherein the nanoparticles are:
tuned to interact with microwaves such that the nanoparticles are more lossy in the presence of microwaves than the cells or tissue of interest are, and
functionalized with a functional coating.

13. The method of claim 12 wherein the functional coating is a biocompatibility coating, an inorganic coating, or a hydrophilic coating.

14. The method of claim 12 wherein the functional coating comprises a targeting ligand and wherein the targeting ligand targets the cell or tissue of interest.

15. The method of claim 12 wherein the functional coating comprises a material that promotes nanoparticle aggregation within the cell or tissue of interest.

16. The method of claim 9 wherein the nanoparticles have diameters of 1-500 nm.

17. A nanoparticle for treating a cell or tissue of interest, wherein the nanoparticle is:
tuned to interact with microwaves such that the nanoparticle is more lossy in the presence of microwaves than the cells or tissue of interest are, and
functionalized with a functional coating.

18. The nanoparticle of claim 17 wherein the functional coating, is a biocompatibility coating, an inorganic coating, or a hydrophilic coating.

19. The nanoparticle of claim 17 wherein the functional coating comprises a targeting ligand and wherein the targeting ligand targets the cell or tissue of interest.

20. The nanoparticle of claim 17 wherein the functional coating comprises a material that promotes nanoparticle aggregation within the cell or tissue of interest.

21. The nanoparticle of claim 17 having a diameter of 1-500 nm.

22. A system for controlling effects of a field of microwave radiation in a cell or tissue of interest in a subject in need thereof comprising:
a source of microwave radiation;
an electronic system for monitoring of the microwave radiation;
a system for delivery of the microwave radiation to the cell or tissue:
microwave-active nanoparticles that absorb the microwave radiation;
an injection or administration system for administration of the nanoparticles;
wherein:
the microwave-active nanoparticles react to microwave energy of the field of microwave radiation by releasing heat, and
the cell or tissue is heated, thereby inducing hyperthermia or thermotherapy in the cell or tissue,
and whereby the effects of the field of microwave radiation are controlled.
